(19) United States
(12) Patent Application Publication
(10) Pub. No.: US 2010/0130882 A1
(43) Pub. Date: May 27, 2010

(54) APPARATUS, METHOD AND PROGRAM FOR ADJUSTING DISTINCTION METHOD FOR ELECTROENCEPHALOGRAM SIGNAL

(76) Inventors: Toru NAKADA, Kyoto (JP); Koji Morikawa, Kyoto (JP)

Correspondence Address: MARK D. SARALINO (PAN);RENNER, OTTO, BOISSELLE & SKLAR, LLP, 1621 EUCLID AVENUE, 19TH FLOOR, CLEVELAND, OH, 44115, US

(21) Appl. No.: 12/634,083

(22) Filed: Dec. 9, 2009

Related U.S. Application Data
(63) Continuation of application No. PCT/JP09/01855, filed on Apr. 23, 2009.

(30) Foreign Application Priority Data
May 15, 2008 (JP) 2008-128866

(51) Int. Cl. A61B 5/0476

(52) U.S. Cl. 600/544

(57) ABSTRACT

The electroencephalogram distinction method adjustment apparatus is used for adjusting a distinction method in an electroencephalogram interface section which™ is provided in an electroencephalogram interface system. The apparatus includes a category determination section and a distinction method adjustment section. The category determination section prestores reference data for classifying a characteristic feature of an electroencephalogram signal, and determines which one of a plurality of classified categories the measured electroencephalogram signal belongs to by using the reference data and a characteristic amount which is common to electroencephalogram signals with respect to two or more options presented by the output section. Based on the result of categorization, the distinction method adjustment section adjusts the distinction method for an electroencephalogram signal with respect to the option selected by the user.

This is a continuation of International Application No. PCT/JP2009/001855, with an international filing date of Apr. 23, 2009, which claims priority of Japanese Patent Application No. 2008-128866, filed on May 15, 2008, the contents of which are hereby incorporated by reference.

### BACKGROUND OF THE INVENTION

1. Field of the Invention

The present invention relates to an interface (electroencephalogram interface) system which makes it possible to manipulate a device by utilizing an electroencephalogram. More specifically, the present invention relates to a device which realizes a function of adjusting a distinction method for an electroencephalogram in an electroencephalogram interface system in order to precisely analyze electroencephalograms which significantly differ from individual person to person.

2. Description of the Related Art

In recent years, various types of information devices such as television sets, mobile phones, and PDAs (Personal Digital Assistants) have become prevalent and entered into people's lives, and thus a user needs to manipulate information devices in many scenes of usual life. Usually, a user manipulates an information device by employing an input means (interface section) such as pressing a button, moving a cursor and making a decision, or manipulating a mouse while watching the screen. However, in the case where both hands are unavailable due to any work other than the device manipulations, e.g., household chores, rearing of children, or driving, it may be difficult to make an input by utilizing an interface section, thus rendering the device manipulation impossible. This has promoted the user's need to manipulate an information device in all kinds of situations.

In answer to such needs, input means utilizing biological signals from a user has been developed. For example, Non-Patent document 1 (Emanuel Donchin and two others, “The Mental Prosthesis: Assessing the Speed of a P300-Based Brain-Computer Interface”, IEEE TRANSACTIONS ON REHABILITATION ENGINEERING, Vol. 8, No. 2, June 2000) discloses an electroencephalogram interface technique that utilizes an event-related potential of an electroencephalogram for distinguishing an option which a user wishes to select. To specifically describe the technique described in Non-Patent document 1, options are randomly highlighted, and the waveform of an event-related potential which appears near about 300 milliseconds since the timing of highlighting an option as a starting point it utilized to enable distinction of an option which the user wishes to select. According to this technique, even in a situation where both hands of a user are full, or even in a situation where the user is unable to move his or her limbs due to an illness or the like, the user can select an option which they wish to select, whereby an interface for device manipulations, etc., that satisfies the aforementioned needs is realized.

As used herein, an “event-related potential” refers to a transient potential fluctuation in the brain which occurs in temporal relationship with an external or internal event. An electroencephalogram interface utilizes this event-related potential as measured from a starting point which is the point in time when an external event occurs. For example, selection of an menu option is supposed to be possible by utilizing a component of an event-related potential called “P300” which occurs in response to a visual stimulation or the like. Generally speaking, “P300” is often regarded as a positive component of an event-related potential which appears near about 300 milliseconds after a starting point, irrespective of the type of sensory stimulation such as auditory sense, visual sense, or somatic sensation.

In order to realize an interface application of an event-related potential, it is important to distinguish the event-related potential of interest (e.g. P300 component) with a high accuracy. Therefore, it is necessary to accurately measure a biological signal, and accurately distinguish the measured biological signal with an appropriate distinction technique.

Since there are large individual differences in the manner in which the aforementioned electroencephalogram waveform may appear, it is necessary to realize a highly accurate distinction that supports such individual differences, in order to utilize an event-related potential as an input means for an interface. FIG. 19 shows a diagram which is shown on page 32 of Non-Patent document 2 (Hiroshi NITTONO, “Event-Related Potential Guidebook For Psychology Research”, KITAOJI SHOBO, issued on Sep. 20, 2005, p 32). FIG. 19 illustrates examples of individual differences in electroencephalograms when a discrimination problem with respect to visual stimulations is presented to 36 test subjects. In the graph of each test subject, electroencephalograms for two kinds of situations are presented, as shown by a solid line and a broken line. As is clear from FIG. 19, it can be said that it is difficult to accurately perform distinction for every user by relying on a single criterion, because there is great variation in the waveform and amplitude at the peak position, due to individual differences.

One conceivable method of accurately distinguishing electroencephalograms having large individual differences is to perform a system adjustment with respect to each user in advance (so-called calibration). This will be specifically described with reference to Portion (a) of FIG. 20. Portion (a) of FIG. 20 shows a procedure of calibration. Before using an electroencephalogram interface, a user is asked to perform a task of manipulating an imaginary electroencephalogram interface. For example, when a user is asked to perform a task of selecting one option from among four options by using an electroencephalogram interface, the four options are highlighted consecutively or randomly, and four pieces of electroencephalogram waveform data are obtained, based on the timing with which an option is highlighted as a starting point (step 41). At the same time, correct answer data as to which option (target option) the user was wishing to select (step 42) is also obtained. Then, by using a characteristic feature of the electroencephalogram waveform data for the target option described in the correct answer data, an adjustment is made with respect to each user to arrive at an optimum distinction method (step 43), and with this adjusted distinction method, an option which the user wishes to select when actually using an electroencephalogram interface is distinguished (step 44).

For example, Patent document 1 (Japanese Laid-Open Patent Publication No. 2005-34620) discloses a technique which, by taking into account the individual differences that appear in a component of an event-related potential, adjusts the distinction method with respect to each user for an improved distinction rate. Instead of performing a distinction for every user with a single criterion, from each user's electroencephalogram acquired through an advance calibration, this technique extracts and stores an optimum component of an event-related potential for each user upon distinction, and distinguishes an option which the user wishes to select by using this component. In addition to the P300 component, the P200 component, the N200 component, or a combination thereof is mentioned as an optimum component of an event-related potential of each user. In Patent document 1, the P200 component is defined as a positive component of an event-related potential which appears near about 200 milliseconds from a starting point, whereas the N200 component is defined as a negative component of an event-related potential which appears near about 200 milliseconds from a starting point.

However, in Patent document 1, as an experiment for extracting and storing individual differences, 100 experiments are carried out per test subject (paragraph 0050). Since it is described that one experiment takes about 1 minute of time, about 100 minutes of time is required for the entire calibration. For example, when a user having purchased a certain consumer device actually uses it, the user will be suffering a large burden and taking troubles if a calibration that requires about 100 minutes of time in advance must be performed.

As opposed to a device which is owned by an individual person, when an electroencephalogram interface is to be applied to a system which is utilized by an indefinite number of users or to a system which permits a limited time of use, e.g., a ticket vending machine at a train station, a bank ATM, or a waiting system of a hospital, it will be a burden on the users, and very inefficient and unpractical from the standpoint of system administration, to perform a time-consuming calibration for each user who uses it.

Therefore, when an electroencephalogram interface is incorporated in a consumer device or applied to a system which is used by an indefinite number of users, the trouble of calibration must be eliminated so as to allow the user to easily use it, and allow the electroencephalogram interface to accurately operate and exhibit the expected functions.

On the other hand, a technique has been developed which categorizes measured electroencephalogram waveform data according to a previously-prepared category system, and determines a process based on the result of categorization. For example, in Patent document 2 (Japanese Laid-Open Patent Publication No. 7-108848), from the electroencephalogram waveform data of a driver, the numbers of a waves, fast waves, and slow waves per unit time are calculated, and based on these values, a categorization is made into one of “normal”, “hazy”, “slightly drowsy”, and “hypnagogic”, which define a previously-prepared category system. Then, according to the result of categorization, a process is determined among “no stimulation”, “stimulation (scent)”, “stimulation (air pressure)”, and “stimulation (buzzer sound)”.

In an electroencephalogram interface for selecting a device manipulation, in order to eliminate the user's burden of calibration and enable accurate distinction, a method might be possible which makes a categorization into one type in a previously-prepared category system based on electroencephalogram waveform data, and adjusts the distinction method based on the result of categorization.

However, such a method will have a problem. This problem is described with reference to Portion (b) of FIG. 20. Portion (b) of FIG. 20 shows a procedure of performing a calibration by categorizing the electroencephalogram waveform data of a user. For example, rather than at an advance calibration, when a user is actually going to select one option from among four options by using an electroencephalogram interface, four pieces of electroencephalogram waveform data will be obtained (step 45). It is assumed that these four pieces of electroencephalogram waveform data include one piece of electroencephalogram waveform data corresponding to an option which the user wanted to select (target option) and three pieces of electroencephalogram waveform data corresponding to other options (non-target options). From these pieces of electroencephalogram waveform data, a categorization into one type in a previously-prepared category system is made (step 46), and according to the result of categorization, an adjustment for arriving at an optimum distinction method is made (step 47), and with the adjusted distinction method, the option which the user wishes to select is distinguished (step 48).

The aforementioned type category (step 46) needs to be a category that reflects a characteristic feature of the electroencephalogram waveform data corresponding to the target option, among the electroencephalogram waveform data corresponding to the respective options (i.e., four pieces of electroencephalogram waveform data in the example of Portion (b) of FIG. 20). The reason is that, if the category reflects a characteristic feature of any other electroencephalogram waveform data, the subsequent process of adjusting the distinction method for accurately distinguishing the target option will not be precisely performed. This is also clear from the fact that, if the correct answer data is not input in the example of Portion (a) of FIG. 20, i.e., if a characteristic feature of the electroencephalogram waveform data corresponding to the target option is not correctly extracted, a precise adjustment of the distinction method cannot be made.

However, when actually using an electroencephalogram interface, there is no correct answer data as to which one is the electroencephalogram waveform data corresponding to the target option, and therefore the electroencephalogram waveform data corresponding to the target option cannot be identified at the point of performing the aforementioned type categorization. Therefore, the type categorization and the adjustment of the distinction method cannot be precisely performed, thus making it impossible to maintain a high accuracy of distinction. Thus, in order to precisely perform a type categorization and an adjustment of the distinction method, it is necessary to infer a characteristic feature of the electroencephalogram waveform data corresponding to the target option, from the electroencephalogram waveform data corresponding to a plurality of options among which the target option cannot be identified.

The aforementioned problem will not be problematic when, as in the conventional technique of Patent document 2, the electroencephalogram waveform data is not to be utilized after categorization. On the other hand, it will be problematic when the electroencephalogram waveform data is to be utilized after categorization, in order to distinguish a target option based on the result of categorization, as described above.

### SUMMARY OF THE INVENTION

An objective of the present invention is to, in the case where electroencephalogram waveform data is utilized for distinguishing a target option, precisely perform a type categorization and an adjustment of the distinction method based on an electroencephalogram waveform of a user, thus freeing the user from the burden of a complicated calibration and yet maintaining a high accuracy of distinction concerning the electroencephalogram.

In an electroencephalogram interface system having an output section for presenting on a screen a plurality of options related to device operations and highlighting each option, an electroencephalogram measurement section for measuring an electroencephalogram signal from a user, and an electroencephalogram interface section for, by using a previously determined distinction method, distinguishing an event-related potential with respect to an option which the user wishes to select from an event-related potential of the electroencephalogram signal based on a timing of highlighting each option as a starting point, and determining an operation of a device, an adjustment apparatus according to the present invention is used for adjusting the distinction method in the electroencephalogram interface section. The distinction method is a method of distinguishing a component of the event-related potential based on whether the electroencephalogram signal satisfies a predetermined criterion or not. The apparatus comprises: a category determination section which prestores reference data for classifying a characteristic feature of an electroencephalogram signal, for determining which one of a plurality of classified categories the measured electroencephalogram signal belongs to by using the reference data and a characteristic amount which is common to electroencephalogram signals with respect to two or more options presented by the output section; and a distinction method adjustment section for, based on a result of categorization, adjusting the distinction method for an electroencephalogram signal with respect to the option selected by the user.

The electroencephalogram signals with respect to the two or more options presented by the output section used by the category determination section may be electroencephalogram signals with respect to all of the plurality of options that are presented by the output section.

As the characteristic amount which is common to electroencephalogram signals with respect to all of the plurality of options, the category determination section may retain an average value of power spectrum values of a predetermined frequency band and/or an average value of wavelet coefficients of a predetermined time period and frequency band of the electroencephalogram signals with respect to the two or more options presented by the output section.

The category determination section may determine an amplitude of an N200 component of the electroencephalogram signal by using an average value of power spectrum values in a frequency band from 8 Hz to 15 Hz.

The category determination section may determine an amplitude of a P200 component of the electroencephalogram signal by using an average value of wavelet coefficients of a time period from 200 milliseconds to 250 milliseconds and a frequency band from 8 Hz to 15 Hz.

Based on the result of categorization, the distinction method adjustment section may adjust weighting factors for a P300 component, a P200 component, and an N200 component of an electroencephalogram signal, the weighting factors being used for distinguishing an electroencephalogram signal with respect to the option selected by the user.

For each of the plurality of classified categories, the distinction method adjustment section may retain a template to be used for distinguishing an electroencephalogram signal with respect to the option selected by the user, and the distinction method adjustment section may adjust the distinction method for the electroencephalogram signal by using a template corresponding to the result of categorization.

By adopting model data to be used for distinguishing an electroencephalogram signal with respect to the option selected by the user according to the result of categorization, the distinction method adjustment section may adjust the distinction method for the electroencephalogram signal.

In an electroencephalogram interface system having an output section for presenting on a screen a plurality of options related to device operations and highlighting each option, an electroencephalogram measurement section for measuring an electroencephalogram signal from a user, and an electroencephalogram interface section for, by using a previously determined distinction method, distinguishing an event-related potential with respect to an option which the user wishes to select from an event-related potential of the electroencephalogram signal based on a timing of highlighting each option as a starting point, and determining an operation of a device, an adjustment method according to the present invention is used for adjusting the distinction method in the electroencephalogram interface section. The distinction method is a method of distinguishing a component of the event-related potential based on whether the electroencephalogram signal satisfies a predetermined criterion or not. The method comprises the steps of: preparing reference data for classifying a characteristic feature of an electroencephalogram signal; determining which one of a plurality of classified categories the measured electroencephalogram signal belongs to by using the reference data and a characteristic amount which is common to electroencephalogram signals with respect to two or more options presented by the output section; and based on a result of categorization, adjusting the distinction method for an electroencephalogram signal with respect to the option selected by the user.

In an electroencephalogram interface system having an output section for presenting on a screen a plurality of options related to device operations and highlighting each option, an electroencephalogram measurement section for measuring an electroencephalogram signal from a user, and an electroencephalogram interface section for, by using a previously determined distinction method, distinguishing an event-related potential with respect to an option which the user wishes to select from an event-related potential of the electroencephalogram signal based on a timing of highlighting each option as a starting point, and determining an operation of a device, a computer program according to the present invention is embodied in a computer-readable medium and is used for adjusting the distinction method in the electroencephalogram interface section. The distinction method is a method of distinguishing a component of the event-related potential based on whether the electroencephalogram signal satisfies a predetermined criterion or not. The computer program causes a computer which is provided in the electroencephalogram interface system to execute the steps of: prestoring reference data for classifying a characteristic feature of an electroencephalogram signal; determining which one of a plurality of classified categories the measured electroencephalogram signal belongs to by using the reference data and a characteristic amount which is common to electroencephalogram signals with respect to two or more options presented by the output section; and based on a result of categorization, adjusting the distinction method for an electroencephalogram signal with respect to the option selected by the user.

In an electroencephalogram interface system having an output section for presenting on a screen a plurality of options related to device operations and highlighting each option, an electroencephalogram measurement section for measuring an electroencephalogram signal from a user, and an electroencephalogram interface section for, by using a previously determined distinction method, distinguishing an event-related potential with respect to an option which the user wishes to select from an event-related potential of the electroencephalogram signal based on a timing of highlighting each option as a starting point, and determining an operation of a device, an adjustment apparatus according to the present invention is used for adjusting the distinction method in the electroencephalogram interface section. The distinction method is a method of distinguishing a component of the event-related potential based on whether the electroencephalogram signal satisfies a predetermined criterion or not. The apparatus comprises: a characteristic amount extraction section for (i) selecting electroencephalogram signals with respect to two or more options from among the electroencephalogram signals with respect to the options, and (ii) prestoring reference data, and extracting a characteristic amount which is common to the reference data and the selected electroencephalogram signals; and a distinction method adjustment section for adjusting the distinction method for an electroencephalogram signal with respect to the option selected by the user so as to apply weighting according to the extracted characteristic amount in distinguishing the electroencephalogram signal with respect to the option selected by the user.

In an electroencephalogram interface system having an output section for presenting on a screen a plurality of options related to device operations and highlighting each option, an electroencephalogram measurement section for measuring an electroencephalogram signal from a user, and an electroencephalogram interface section for, by using a previously determined distinction method, distinguishing an event-related potential with respect to an option which the user wishes to select from an event-related potential of the electroencephalogram signal based on a timing of highlighting each option as a starting point, and determining an operation of a device, a method according to the present invention is used for adjusting the distinction method in the electroencephalogram interface section. The distinction method is a method of distinguishing a component of the event-related potential based on whether the electroencephalogram signal satisfies a predetermined criterion or not. The method comprises the steps of: selecting electroencephalogram signals with respect to two or more options from among the electroencephalogram signals with respect to the options; prestoring reference data, and extracting a characteristic amount which is common to the reference data and the selected electroencephalogram signals; and adjusting the distinction method for an electroencephalogram signal with respect to the option selected by the user so as to apply weighting according to the extracted characteristic amount in distinguishing the electroencephalogram signal with respect to the option selected by the user.

In an electroencephalogram interface system having an output section for presenting on a screen a plurality of options related to device operations and highlighting each option, an electroencephalogram measurement section for measuring an electroencephalogram signal from a user, and an electroencephalogram interface section for, by using a previously determined distinction method, distinguishing an event-related potential with respect to an option which the user wishes to select from an event-related potential of the electroencephalogram signal based on a timing of highlighting each option as a starting point, and determining an operation of a device, a computer program according to the present invention is used for adjusting the distinction method in the electroencephalogram interface section. The distinction method is a method of distinguishing a component of the event-related potential based on whether the electroencephalogram signal satisfies a predetermined criterion or not. The computer program causes a computer which is provided in the electroencephalogram interface system to execute the steps of: selecting electroencephalogram signals with respect to two or more options from among the electroencephalogram signals with respect to the options; prestoring reference data, and extracting a characteristic amount which is common to the reference data and the selected electroencephalogram signals; and adjusting the distinction method for an electroencephalogram signal with respect to the option selected by the user so as to apply weighting according to the extracted characteristic amount in distinguishing the electroencephalogram signal with respect to the option selected by the user.

According to the present invention, in a system having an interface for distinguishing from among a plurality of options an option which a user wishes to select by utilizing an electroencephalogram, by using a characteristic amount(s) which is common to electroencephalogram signals corresponding to all options, a categorization into one type in a previously-prepared category system is made. Based on the result of categorization, an adjustment for arriving at an optimum distinction method is made.

As a result, it is unnecessary to perform a calibration for the user, so that the user's burden and cumbersomeness can be greatly reduced, and yet, a high accuracy of distinction can be maintained by adjusting the distinction method based on the categorized type.

Other features, elements, processes, steps, characteristics and advantages of the present invention will become more apparent from the following detailed description of preferred embodiments of the present invention with reference to the attached drawings.

### BRIEF DESCRIPTION OF THE DRAWINGS

FIG. 1 is a diagram showing a construction and an environment of use of an electroencephalogram interface system 1 in an example where a television set and a wearable-type electroencephalograph are combined.

FIG. 2 is a diagram showing a functional block construction of an electroencephalogram interface system 1 according to Embodiment 1.

FIG. 3 is a flowchart showing a procedure of processing by the electroencephalogram interface 1.

Portions (a) to (d) of FIG. 4 are diagrams showing transitions of screens when a user 10 selects a program of a genre that the user 10 wishes to view in the electroencephalogram interface system 1.

FIG. 5 is a diagram showing waveforms obtained by taking an arithmetic mean, with respect to each test subject, of electroencephalogram waveform data obtained from test subjects 01 to 13 through an experiment.

FIG. 6 is a diagram showing a category system concerning the electroencephalogram waveform data of each test subject shown in FIG. 5, in which characteristic features of the electroencephalograms of individual people are classified based on the levels of the P200 component and the N200 component before 300 milliseconds.

Portions (a) to (d) of FIG. 7 are diagrams showing total arithmetic mean waveforms of electroencephalogram waveform data of respective types of categorization.

FIG. 8 is a diagram showing power spectrum values of electroencephalogram waveform data concerning a test subject group (7 people) whose N200 component is “Large” and a test subject group (6 people) whose N200 component is “Small” in the category system shown in FIG. 6.

FIG. 9 is a diagram in which a relationship between the “Large”, “Middle”, and “Small” levels of the P200 component in the category system shown in FIG. 6 and wavelet coefficients of electroencephalogram waveform data, given a predetermined time frequency component and frequency band, is plotted with respect to each test subject.

FIG. 10 is a diagram showing the procedure of a categorization process by a category determination section 14.

FIG. 11 is a diagram showing a portion of reference data for type categorization which is generated based on experimental results.

FIG. 12 is a flowchart showing a procedure of processing by a distinction method adjustment section 15.

FIG. 13 is a diagram showing weighting factors for the P300 component, the P200 component, and the N200 component with respect to each type.

Portions (a) and (b) of FIG. 14 are diagrams showing examples of model data for type A.

FIG. 15 is a diagram showing average values, across all test subjects, of the distinction rate of a target option under three conditions.

FIG. 16 is a diagram showing distinction rates of test subjects of type A, test subjects of type D, and other test subjects, as a breakdown of FIG. 15.

FIG. 17 is a diagram showing distinction rates of test subjects of type A and type D, with respect to the three conditions where the characteristic amounts to be used for type categorization are: (b) both power spectrum values and wavelet coefficients; (b-1) only power spectrum values; and (b-2) only wavelet coefficients.

FIG. 18 is a diagram showing a functional block construction of an electroencephalogram interface system 3 according to Embodiment 2.

FIG. 19 is a diagram showing exemplary individual differences in electroencephalograms when a discrimination problem with respect to visual stimulations is presented to 36 test subjects.

Portion (a) of FIG. 20 is a diagram showing a procedure of calibration; and portion (b) of FIG. 20 is a diagram showing a procedure of calibration which involves categorizing electroencephalogram waveform data of a user.

### DETAILED DESCRIPTION OF PREFERRED EMBODIMENTS

Hereinafter, with reference to the attached drawings, embodiments of the electroencephalogram interface system and electroencephalogram distinction method adjustment apparatus according to the present invention will be described.

First, an outline of main characteristic features of the electroencephalogram interface system and electroencephalogram distinction method adjustment apparatus according to the present invention will be described. Thereafter, respective embodiments of the electroencephalogram interface system will be described.

The inventors envisage that, in future, an electroencephalogram interface system will be constructed in an environment in which a wearable-type electroencephalograph and a wearable-type display are combined. The user will always be wearing the electroencephalograph and the display, and be able to perform content viewing and screen manipulation by using the wearable-type display. Otherwise, it is envisaged that an electroencephalogram interface system will be constructed in an environment (e.g., home) in which a home television set and a wearable-type electroencephalograph are combined. When watching television, the user is able to perform content viewing and screen manipulation by wearing the electroencephalograph.

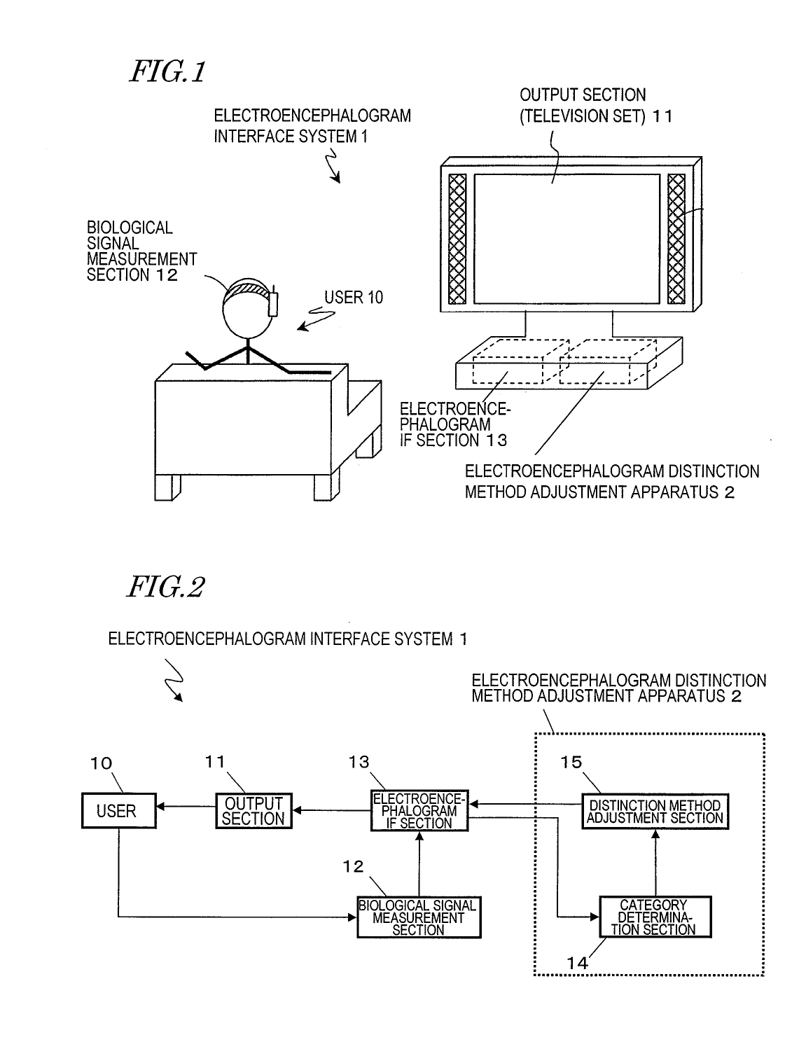

For example, FIG. 1 shows a construction and an environment of use for the electroencephalogram interface system 1 as envisaged by the inventors in the latter example. The electroencephalogram interface system 1 is exemplified so as to correspond to a system construction of Embodiment 1 described later.

The electroencephalogram interface system 1 is a system for providing an interface for manipulating a television set 11 by utilizing an electroencephalogram signal from a user 10. When a plurality of options that are displayed on the television set 11 are highlighted one by one, an influence will appear in an event-related potential of the electroencephalogram of the user 10 since each highlighting as a starting point. An electroencephalogram signal from the user 10 is acquired by an electroencephalogram measurement section 12 which is worn on the head of the user, and transmitted to an electroencephalogram IF section 13 in a wireless or wired manner. The electroencephalogram IF section 13 internalized in the television set 11 recognizes an option which the user wishes to select by utilizing an event-related potential of the electroencephalogram of the user 10. As a result, it becomes possible to perform a process, such as channel switching, in accordance with the intent of the user.

In the electroencephalogram interface (IF) section (described later) of the electroencephalogram interface system 1, a predetermined distinction method is previously defined. This “distinction method” is a method of distinguishing a component of an event-related potential depending on whether an electroencephalogram signal satisfies a previously determined criterion or not.

In order to distinguish an option which the user wishes to select, it is necessary to optimize the distinction method depending on the user, by utilizing an event-related potential of the electroencephalogram of the user 10.

In the present embodiment, from electroencephalogram waveform data, the electroencephalogram distinction method adjustment apparatus 2 internalized in the television set 11 categorizes a characteristic feature of the electroencephalogram of an individual person into one type in a classified category system, and in accordance with the result of categorization, performs a process of adjusting the distinction method used in the electroencephalogram IF section 13 to be optimum. At this time, a characteristic amount(s) which is common to electroencephalogram signals corresponding to all options, rather than only an electroencephalogram signal of when a specific option is highlighted, is used. Corresponding to the previously determined category system, two electroencephalogram waveform templates (model data) are also provided, for example. One is model data to appear when an option which the user wishes to select is highlighted; the other is model data to appear when an option which the user does not wish to select is highlighted. By comparing the resultant electroencephalogram waveform data and each of these model data and making an evaluation as to which one is closer, it can be determined as to whether the user wanted to select the highlighted option when this electroencephalogram waveform is measured.

Although there are large individual differences in the manner in which an electroencephalogram waveform appears, the inventors have found characteristic features that are common to the electroencephalogram waveforms of a plurality of users, and performed categorization with respect to each characteristic feature, while also providing model data for each category that enables distinction of the characteristic feature. As a result, it is possible to adopt a distinction method which is optimum to that user, based on the result of categorization.

The inventors have carried out the categorization by utilizing the N200 component and P200 component (described later) of event-related potentials which were obtained with one stimulation (or a smaller number of stimulations, e.g., only a few) for every option. The inventors have found that it is effective to perform a categorization based on average values of the power spectrum values of a frequency band and average values of the wavelet coefficients of a frequency band.

### Embodiment 1

Hereinafter, embodiments of the present invention will be described in detail.

FIG. 2 shows a functional block construction of an electroencephalogram interface system 1 according to the present embodiment. The electroencephalogram interface system 1 includes an output section 11, an electroencephalogram measurement section 12, an electroencephalogram IF section 13, and an electroencephalogram distinction method adjustment apparatus 2. The electroencephalogram distinction method adjustment apparatus 2 is composed of a category determination section 14 and a distinction method adjustment section 15. The user 10 block is illustrated for convenience of explanation, and is not a constituent element of the electroencephalogram interface system 1 itself.

The output section 11 outputs to the user a content and a menu to be selected in the electroencephalogram interface. Since the television set 11 shown in FIG. 1 is a specific example of the output section, reference numeral “11” will hereinafter be assigned to the output section. The output section 11 would correspond to the display screen in the case where the output substance is a moving picture or a still image, and in the case where the output substance contains audio, a display screen and loudspeakers may together be used as the output section 11.

The electroencephalogram measurement section 12 is an electroencephalograph which detects an electroencephalogram signal by measuring a change in potential on electrodes which are worn on the head of the user 10. The electroencephalograph may be a head-mounted electroencephalograph as shown in FIG. 1. It is assumed that the user 10 has put on the previously electroencephalograph in advance.

Electrodes are disposed on the electroencephalogram measurement section 12 so that, when worn on the head of the user 10, the electrodes come in contact with the head at predetermined positions. The positioning of the electrodes may be, for example, Pz (median parietal), A1 (earlobe), and the nasion of the user 10. However, it will suffice if there are at least two electrodes, and potential measurement will be possible with only Pz and A1, for example. These electrode positions are to be determined based on reliability of signal measurements, wearing ease, and the like.

Thus, the electroencephalogram measurement section 12 is able to measure the electroencephalogram of the user 10. The measured electroencephalograms of the user 10 are sampled so as to be computer-processible, and are sent to the electroencephalogram IF section 13. Note that, in order to reduce the influence of noises which may be mixed in the electroencephalogram, the electroencephalogram to be measured in the electroencephalogram measurement section 12 of the present embodiment are subjected to low-pass filtering at e.g. 15 Hz in advance.

The electroencephalogram IF section 13 presents an interface screen concerning device manipulations to the user via the output section 11, and consecutively or randomly highlights a plurality of options on the interface screen. From the electroencephalogram waveform data measured by the electroencephalogram measurement section 12, the electroencephalogram IF section 13 distinguishes an option which the user wanted to select. Hereinafter, in the present embodiment, an option which the user wanted to select will be referred to as a “target option”, whereas any option other than the target option will be referred to as a “non-target option”.

In the following descriptions, “options” will be described as possible programs that may be being desired for watching (“baseball”, “weather forecast”, “cartoon show”, and “news” in portion (b) of FIG. 4). However, this is only an example. Whenever there are a plurality of items corresponding to selectable manipulations on a device to be manipulated, each item corresponds to an “option” in the sense of the present specification. The manner of displaying an “option” may be arbitrary.

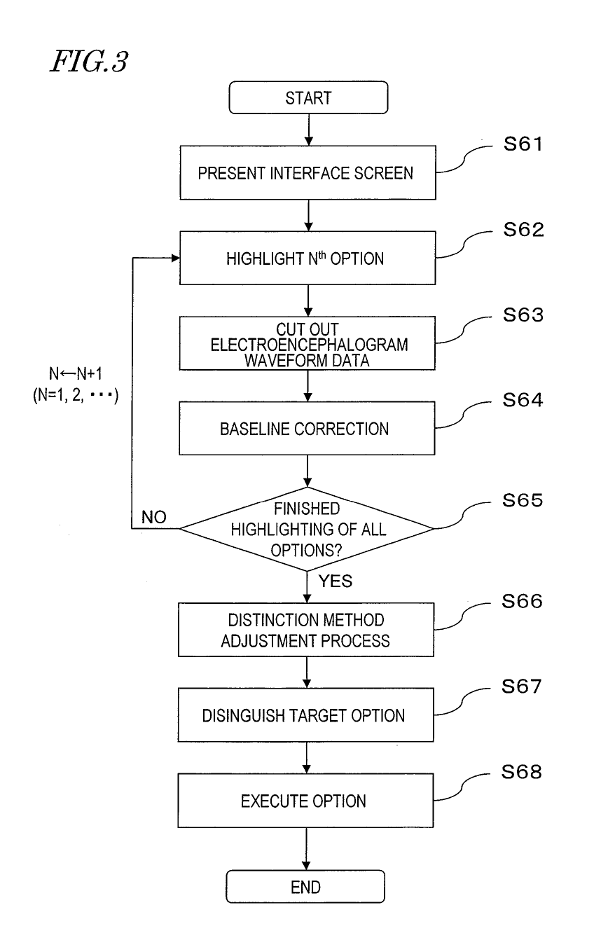

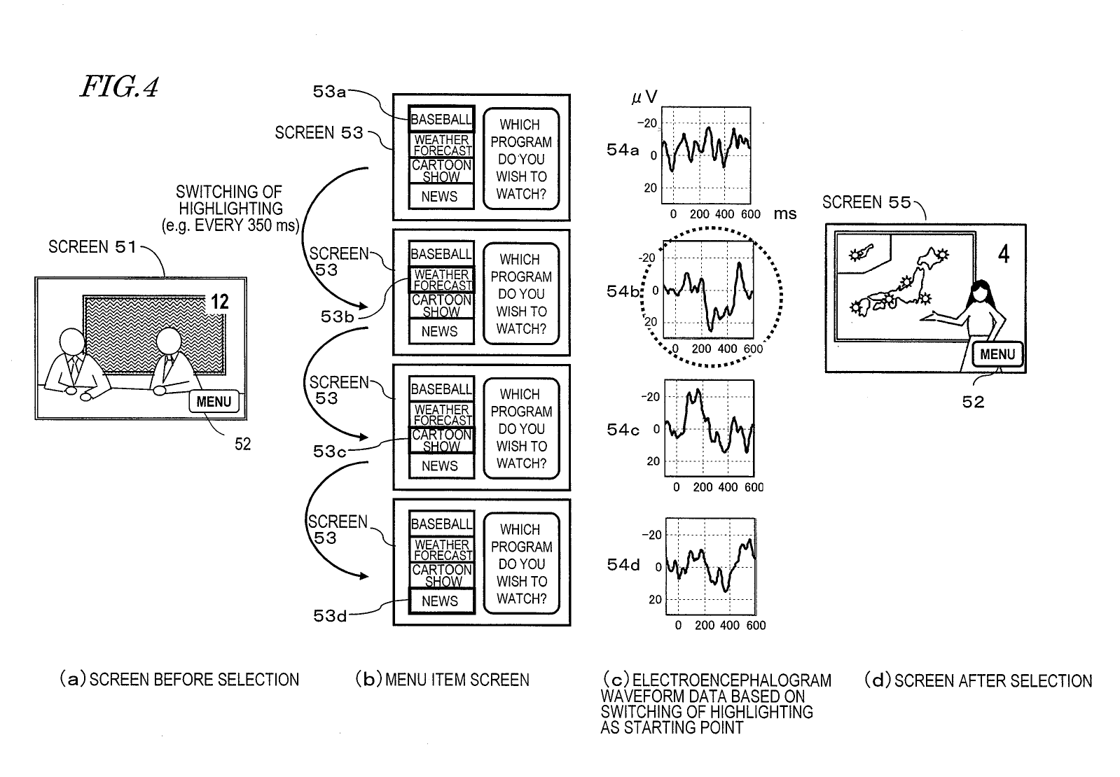

Referring to FIG. 3 and FIG. 4 as necessary, a procedure of processing by the electroencephalogram interface 1 shown in FIG. 2 will be described. FIG. 3 is a flowchart showing a procedure of processing by the electroencephalogram interface system 1. Portions (a) to (d) of FIG. 4 are diagrams showing transitions of screens when the user 10 selects a program of a genre that the user 10 wishes to view in the electroencephalogram interface system 1.

At step S61, the electroencephalogram IF section 13 determines whether or not to activate an electroencephalogram interface by using an SSVEP, and presents an interface screen via the output section 11. An SSVEP means steady state visual evoked potential.

For example, when the user 10 is viewing a content, a screen 51 before selection (which in this case is news) as shown in portion (a) of FIG. 4 may be displayed on the television set. A menu 52 displayed at the lower right is flickering at a specific frequency. When the user 10 watches the menu 52, a specific frequency component is known to be superposed on the electroencephalogram. Therefore, by distinguishing the power spectrum of a frequency component of the flickering period of the electroencephalogram signal, it is possible to determine whether the menu 52 is being watched or not, whereby an electroencephalogram interface can be activated. To activate an electroencephalogram interface means to begin the operation of an interface for allowing a selection, etc., to be made by using an electroencephalogram.

For example, the SSVEP refers to what is described in Xiaorong Gao, “A BCI-Based Environmental Controller for the Motion-Disabled”, IEEE Transaction on Neural Systems and Rehabilitation Engineering, Vol. 11, No. 2, June 2003.

When the electroencephalogram interface is activated, an interface screen 53 as shown in portion (b) of FIG. 4 is displayed. On the screen, a question that says “Which program do you wish to watch?”, and options which are candidates of a program that may be being desired for watching, are presented. In this example, four are being displayed: “baseball” 53a, “weather forecast” 53b, “cartoon show” 53c, and “news” 53d.

FIG. 3 is referred to again. At step S62, the electroencephalogram IF section 13 causes each option on the interface screen 53 to be consecutively or randomly highlighted via the output section 11. Portion (b) of FIG. 4 illustrates an example where “baseball” 53a, “weather forecast” 53b, “cartoon show” 53c, and “news” 53d are consecutively highlighted from the top of the screen 53. The time interval at which the highlight is switched is 350 milliseconds. Highlighting may be a change in at least one of the luminance, hue, and size of an option on the interface screen. An option may be presented with a pointer which employs an auxiliary arrow instead of or in addition to highlighting.

At step S63, from the electroencephalogram signal measured by the electroencephalogram measurement section 12, the electroencephalogram IF section 13 cuts out the electroencephalogram waveform data from −100 milliseconds to 600 milliseconds based on the point of highlighting each option as a starting point.

At step S64, the electroencephalogram IF section 13 applies a baseline correction to the electroencephalogram waveform data having been cut out. For example, the baseline is corrected with an average potential from −100 milliseconds to 0 milliseconds based on the point of highlighting the option as a starting point.

At step S65, the electroencephalogram IF section 13 determines whether highlighting of every option in the interface screen 53 has been finished or not. If not finished, control returns to S62; if finished, control proceeds to S66.

Note that, generally in the studies of event-related potentials, it is often the case that the same option is highlighted N times (e.g., 5 times, 10 times, 20 times) (e.g., in the case where there are four options, highlighting may be performed 4×N times). Then, an arithmetic mean is determined for each same option, followed by a distinction of the target option. As a result of this, random action potentials of the brain can be counteracted, so that an event-related potential having a certain latency and polarity (e.g., P300 component, P200 component, N200 component) can be detected.

Note that, when the same option is highlighted N times (N: an integer equal to or greater than 2), the accuracy of distinction will be increased, but an amount of time corresponding to the number of processes will be required. Therefore, in the case where an indefinite number of users are to use the electroencephalogram interface system 1, each same option may be highlighted only a small number of times (e.g. 2 or 3 times), or highlighted only once. In determining an arithmetic mean for each same option, there is no limitation as to the number of summations (number of highlightings).

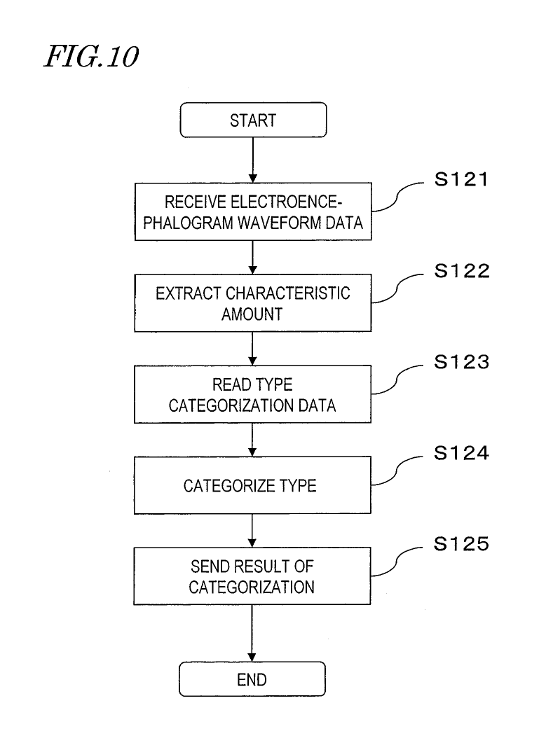

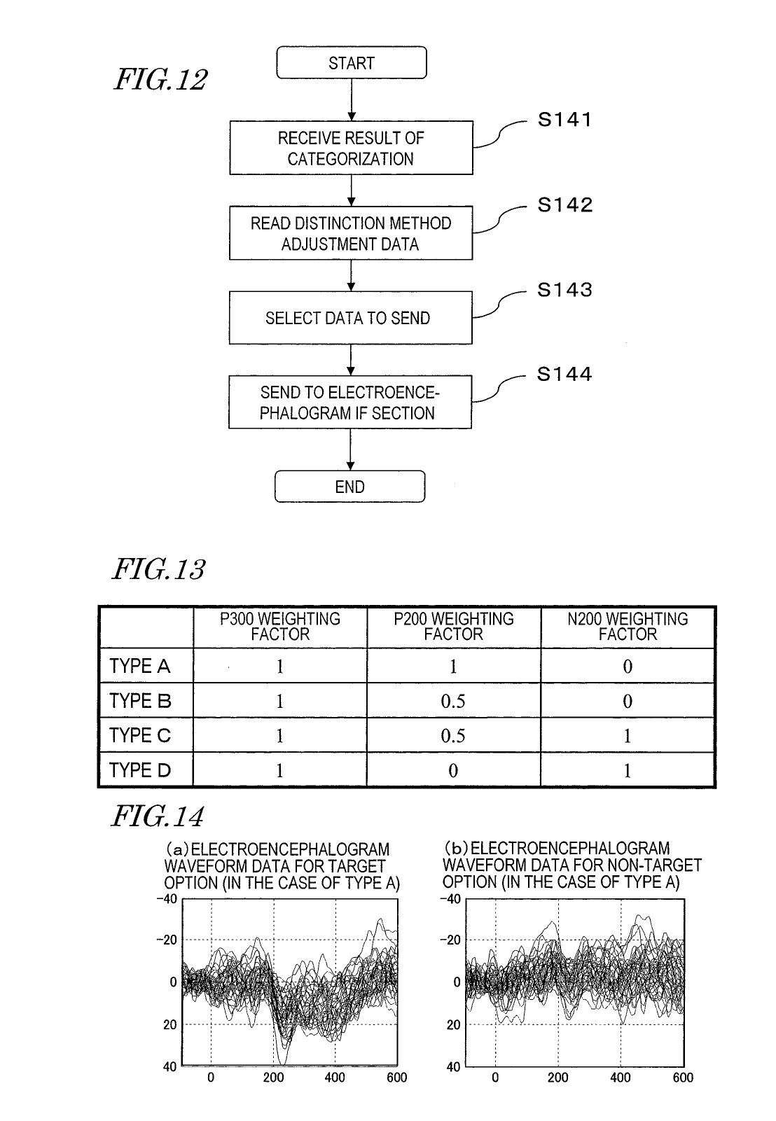

At step S66, by using a characteristic amount(s) which is common to the electroencephalogram waveform data corresponding to all options, the electroencephalogram distinction method adjustment apparatus 2 categorizes a characteristic feature of the electroencephalogram of an individual person into one type in a classified category system, and performs a process of making an adjustment for arriving at an optimum distinction method in accordance with the result of categorization. The details of the process will be described later with reference to the procedures of processing by the category determination section 14 and the distinction method adjustment section 15 shown in FIG. 10 and FIG. 12.

At step S67, in accordance with the type categorization and the result of distinction method adjustment based thereon in the electroencephalogram distinction method adjustment apparatus 2, the electroencephalogram IF section 13 distinguishes a target option from among the plurality of options. For the distinction of the target option, the same signal as the electroencephalogram signal used for the type categorization is used. Since the type categorization and distinction of the option can be performed by using the same electroencephalogram signal, the accuracy of distinction can be improved without performing a calibration which does not involve distinction of an option.

Portion (c) of FIG. 4 shows a manner in which, from among electroencephalogram waveform data 54a to 54d corresponding to four options, the electroencephalogram waveform data 54b is distinguished as the target option. At distinction, the electroencephalogram IF section 13 may make the selection based on the zone average potential of the electroencephalogram waveform data in a given zone, or make the selection based on the value of a correlation coefficient with respect to a template, regarding each highlighted option. Alternatively, the electroencephalogram IF section 13 may make the selection based on a posterior probability value obtained through a linear discriminant analysis or nonlinear discriminant analysis. The details of each of the above methods will be again described following the description of the distinction method adjustment section 15, which performs an adjustment of the distinction method.

At step S68 in FIG. 3, in order to cause an operation of the distinguished option to be executed, the electroencephalogram IF section 13 causes an appropriate device to execute the operation. In the example of portion (d) of FIG. 4, the electroencephalogram IF section 13 instructs the output section (TV) 11 to switch the channel to “weather forecast”, and the output section (TV) 11 is executing that process.

At the process step S66 shown in FIG. 3, the category determination section 14 begins its process upon receiving from the electroencephalogram IF section 13 the electroencephalogram waveform data to be subjected to categorization. In the example of portion (c) of FIG. 4, the electroencephalogram waveform data 54a to 54d corresponding to the four highlighted options are received. Furthermore, by using a characteristic amount(s) which is common to the received electroencephalogram signals corresponding to all options, a characteristic feature of the electroencephalogram of an individual person is categorized into one type in a classified category system. A “characteristic amount which is common to electroencephalogram signals corresponding to all options” means a characteristic feature of waveforms which is obtained by using the electroencephalogram waveforms corresponding to all options. The specific process of calculation will be described later.

In accordance with the result of categorization by the category determination section 14, the distinction method adjustment section 15 makes an adjustment of the distinction method for accurately distinguishing the target option, and sends the result of adjustment to the electroencephalogram IF section 13.

Now, the category system when performing the above-described type categorization will be specifically described based on results of an electroencephalogram interface experiment performed by the inventors.

There were a total of 13 test subjects, including 9 males and 4 females, with an average age of 26±6.5 years. To each test subject, an interface screen containing the four options shown in portion (b) of FIG. 4 was presented on a monitor, and a task was asked which involved looking at the options being highlighted every 350 milliseconds, and thinking “That's it” to themselves immediately after a designated option (target option) was highlighted. Highlighting of the options was repeated a total of 20 times, consisting of 5 times each of the four options in random order (i.e., the number of summations was five times), this constituting one trial of experiment. Moreover, designations of the target options were made in the order of “baseball” 53a, “weather forecast” 53b, “cartoon show” 53c, and “news” 53d from the top, and 10 trials each (a total of 40 trials) of experiment were performed for each test subject.

Moreover, the test subject wore an electroencephalograph (Polymate AP-1124 by TEAC Corporation), with electrodes being positioned according to the International 10-20 electrode method, such that a recording electrode was at Pz (median parietal), a reference electrode was at A1 (right earlobe), and a ground electrode was at the forehead. Electroencephalogram waveform data which was measured with a sampling frequency of 200 Hz and a time constant of 3 seconds was subjected to low-pass filtering at 15 Hz; the electroencephalogram waveform data from −100 milliseconds to 600 milliseconds since highlighting of an option as a starting point was cut out; and a baseline correction was performed with an average potential from −100 milliseconds to 0 milliseconds.

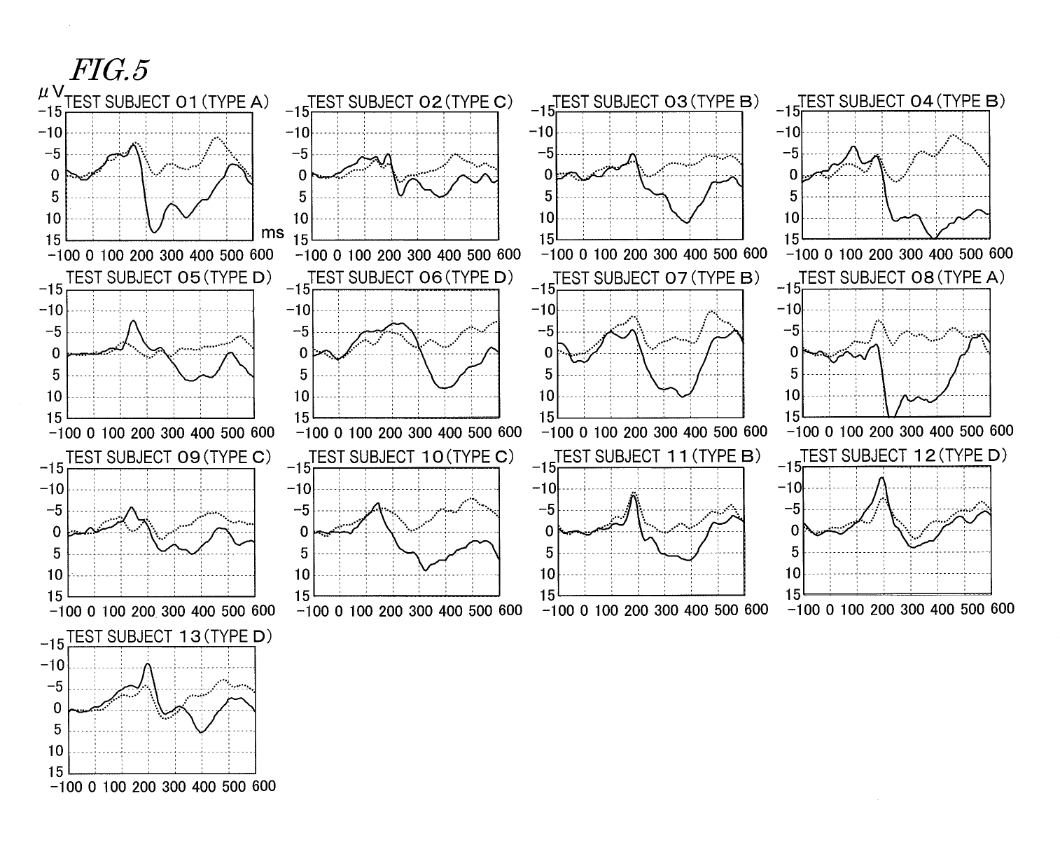

FIG. 5 shows waveforms obtained by taking an arithmetic mean, with respect to each test subject, of electroencephalogram waveform data obtained from test subjects 01 to 13 through the above-described experiment. The horizontal axis represents time (latency) in units of milliseconds, where the highlighting of an option is registered at 0 milliseconds, whereas the vertical axis represents potential in units of μV. Each solid line shows an average waveform of electroencephalogram waveform data for a target option (from 40 trials, with a total number of summations being 40×5=200 times). Each dotted line shows an average waveform of electroencephalogram waveform data for non-target options (from 40 trials for 3 options, with a total number of summations being 3×40×5=600 times).

From the electroencephalogram waveform data of each test subject shown in FIG. 5, there is a common characteristic feature among the electroencephalogram waveform data (solid line) with respect to a target option in that it becomes positive at a latency of 300 milliseconds or later, especially near 400 milliseconds. However, it can be seen that, between 100 milliseconds and 300 milliseconds, the characteristic feature of the electroencephalogram waveform data with respect to a target option differs from test subject to test subject. For example, the electroencephalogram waveform data of test subject 01 with respect to a target option has a large positive component near after 200 milliseconds, whereas the electroencephalogram waveform data test subject 12 with respect to a target option has a large negative component near before 200 milliseconds.

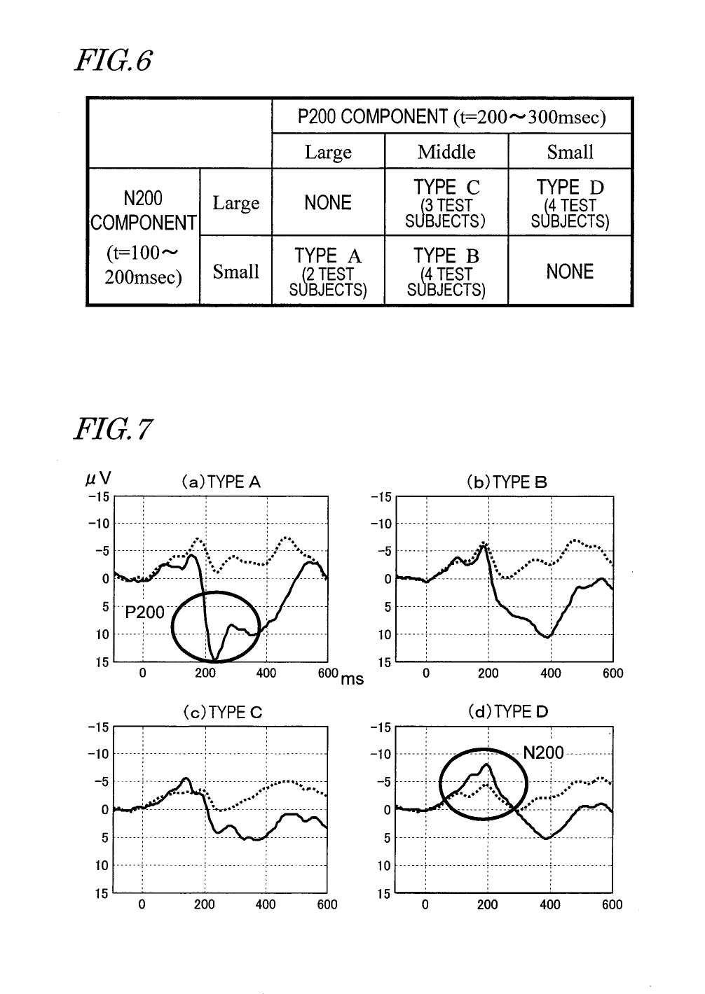

FIG. 6 shows a category system concerning the electroencephalogram waveform data of each test subject shown in FIG. 5, in which characteristic features of the electroencephalograms of individual people are classified based on the levels of the P200 component and the N200 component before 300 milliseconds. The horizontal axis represents the amplitude of the P200 component, whereas the vertical axis represents the amplitude of the N200 component. The levels of the P200 component and the N200 component are determined from both of the target option and the non-target options as shown in FIG. 5.

Specifically, the “P200 component” is obtained by subtracting an average potential of the electroencephalogram waveform with respect to non-target options from 200 milliseconds to 300 milliseconds from an average potential of the electroencephalogram waveform with respect to a target option from 200 milliseconds to 300 milliseconds. If the amplitude of the P200 component thus determined is 10 μV or more, it is designated “Large”; if it is equal to or greater than 1 μV but less than 10 μV, “Middle”; and if it is less than 1 μV, “Small”. The potential thus obtained is an example of a “characteristic amount which is common to electroencephalogram signals corresponding to all options”.

On the other hand, the “N200 component” is obtained by subtracting an average potential of the electroencephalogram waveform data with respect to a target option from 100 milliseconds to 200 milliseconds from an average potential of the electroencephalogram waveform data with respect to non-target options from 100 milliseconds to 200 milliseconds. If the amplitude of the N200 component thus determined is 1.4 μV or more, it is designated “Large”; and if it is less than 1.4 μV, “Small”.

Although 200 milliseconds to 300 milliseconds of the electroencephalogram waveform is chosen for the calculation of the P200 component and the N200 component, this is only an example. For example, 200 milliseconds to 250 milliseconds of the electroencephalogram waveform may be chosen for the calculation of the P200 component. Similarly, although 100 milliseconds to 200 milliseconds of the electroencephalogram waveform is chosen for the calculation of the N200 component, this is only an example.

FIG. 6 also shows a result of categorizing the electroencephalogram waveform data of each test subject shown in FIG. 5 according to the aforementioned categorization criteria. There are 2 test subjects whose P200 component is “Large” and whose N200 component is “Small”, these being designated type A. There are 4 test subjects whose P200 component is “Middle” and whose N200 component is “Small”, these being designated type B. There are 3 test subjects whose P200 component is “Middle” and whose N200 component is “Large”, these being designated type C. There are 4 test subjects whose P200 component is “Small” and whose N200 component is “Large”, these being designated type D. In the present experiment, no test subjects existed whose P200 component and N200 component are both “Large” or both “Small”.

FIG. 7 shows a total arithmetic mean waveform of the electroencephalogram waveform data for each type of categorization above. The horizontal axis represents time (latency) in units of milliseconds, where the highlighting of an option is registered at 0 milliseconds, whereas the vertical axis represents potential in units of μV. Each solid line shows electroencephalogram waveform data with respect to a target option, whereas each dotted line represents electroencephalogram waveform data for non-target options.

From FIG. 7, it can be seen that a large P200 component appears in type A, whereas a large N200 component appears in type D. Based on the electroencephalogram waveform of each user, the category determination section 14 categorizes the waveform into one type in the aforementioned category system.

Furthermore, characteristic amounts to be used for type categorization, which the inventors have newly identified based on results of electroencephalogram interface experiments which they have conducted, will be specifically described. The inventors have conducted various analyses regarding the relationships between: the aforementioned category system based on characteristic features of electroencephalogram waveform data with respect to a target option; and characteristic amounts which are common to the electroencephalogram waveform data of all options. As a result, the inventors have identified two characteristic amounts which have strong correlations. Because of the finding of these characteristic amounts having strong correlations, it is possible to improve the accuracy without performing an advance calibration as in Patent document 1.

That is, it is not necessary to extract and categorize waveform characteristic features with respect to a plurality of target options by performing an advance calibration. It is possible to obtain an improved accuracy by utilizing an electroencephalogram signal with respect to any option, including a target option and non-target options.

Conventionally, a target option would be identified, and a characteristic amount would be extracted from an electroencephalogram waveform with respect thereto. In contrast, characteristic amounts have been found which appear in electroencephalogram waveforms with respect to all options, including non-target options, and thus, without even identifying a target option, an improved accuracy can be obtained by utilizing a user's characteristic feature extracted from the electroencephalogram waveform of any option. This will be described in detail below.

First, FIG. 8 shows power spectrum values of electroencephalogram waveform data of the test subject group whose N200 component is “Large” (7 people) and the test subject group whose N200 component is “Small” (6 people) in the category system shown in FIG. 6. The horizontal axis represents frequency in units of Hz, whereas the vertical axis represent power spectrum values in units of (μV)²/Hz. From the electroencephalogram waveform data which is in chronological order, frequency component data is obtained through Fourier transform. A power spectrum value is calculated as a product of frequency component data and a complex conjugate thereof.

The solid line in FIG. 8 shows the test subject group whose N200 component is “Large”. On the solid line, any “◯” indicates an average value of the power spectra of all electroencephalogram waveform data of the 7 people, including a target option and non-target options, with the upper and lower arrows through the “◯” representing fluctuations among test subjects. The dotted line shows the test subject group whose N200 component is “Small”. On the broken line, any “×” indicates an average value of the power spectra of all electroencephalogram waveform data of the 6 people, including a target option and non-target options, with the upper and lower arrows through the “×” representing fluctuations among test subjects.

At each frequency in FIG. 8, a t test was performed for the “Large” test subject group and the “Small” test subject group. A t test is a statistical significance test. Thus, it has been found that, in the frequency zone from 8 Hz to 15 Hz, average values of the power spectrum values of all electroencephalogram waveform data including the target option and non-target options are significantly lower in the test subject group whose N200 component is “Large” than in the test subject group whose N200 component is “Small” (level of significance P=0.05). The fact that there is a significant difference with a 5% level of significance means that there is a meaningful difference between the data of the two groups with a statistical reliability of 95%.

By utilizing the above relationship, even if a piece of electroencephalogram waveform data that corresponds to the target option is not identified among electroencephalogram waveform data corresponding to a plurality of options, a test subject can be categorized into one whose N200 component is “Large” or one whose N200 component is “Small”, based on the average values of the power spectrum values of all electroencephalogram waveform data in the aforementioned frequency band.

In the example of FIG. 8, in the frequency zone in the neighborhood from 8 Hz to 15 Hz, the test subjects whose N200 component is “Large” and the test subjects whose N200 component is “Small” have average power spectrum values of 1.6 and 3.6, respectively. Therefore, for example, a middle value therebetween, i.e., 2.6, may be chosen as a threshold value. If less than the threshold value of 2.6, a test subject will be determined as “Large”; if equal to or greater than the threshold value of 2.6, a test subject will be determined as “Small”. In the example of FIG. 6, a categorization can be made between the test subjects of type A or B and the test subjects of type C or D. Note that this method of determining a threshold value is exemplary. Other than a middle value, any value may be used that exists between 1.6 and 3.6 as in the above example.

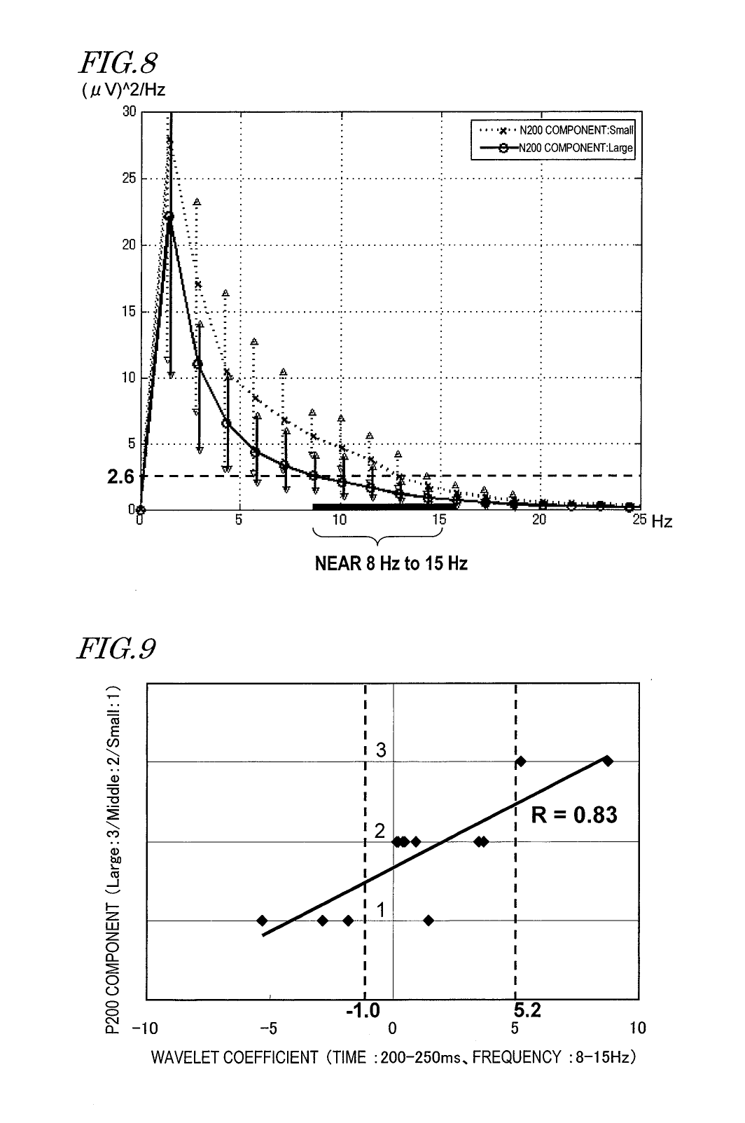

Next, FIG. 9 shows a test-subject-by-test-subject plotting of the relationship between: the “Large”, “Middle”, and “Small” levels of the P200 component in the category system shown in FIG. 6; and a time frequency component of electroencephalogram waveform data, specifically, a wavelet coefficient pertaining to a time period from 200 milliseconds to 250 milliseconds and a frequency band in the neighborhood from 8 Hz to 15 Hz. The wavelet coefficient is of the case where the mother wavelet is a Mexican hat. The vertical axis represents the P200 component level; if “Large”, it is indicated as 3 (corresponding to 2 test subjects); if “Middle”, 2 (corresponding to 7 test subjects); and if “Small”, 1 (corresponding to 4 test subjects). The horizontal axis represents an average value of the wavelet coefficients of all electroencephalogram waveform data, including the target option and non-target options, for each test subject.

A linear regression analysis performed in FIG. 9 resulted in an approximation with the approximate expression y=0.1586x+1.6673, thus indicating that there is a strong correlation between the level (y) of the P200 component and the wavelet coefficient (x) (correlation coefficient R=0.83). A correlation coefficient is a statistical index that indicates a correlation between two variables (degree of similarity). Generally speaking, there is a strong correlation when the correlation coefficient has an absolute value of 0.7 or more.

By utilizing the above relationship, even if the electroencephalogram waveform data corresponding to a target option cannot be identified among electroencephalogram waveform data corresponding to a plurality of options, it is possible to categorize a test subject to be one whose P200 component is “Large”, one whose P200 component is “Middle”, or one whose P200 component is “Small”, based on an average value of the wavelet coefficients with respect to all electroencephalogram waveform data pertaining to the aforementioned time period and frequency band.

In the example of FIGS. 9, x=5.2 and −1.0, which respectively correspond to the levels (y)=2.5 (i.e., a middle value between “Large: 3” and “Middle: 2”) and 1.5 (i.e., a middle value between “Middle: 2” and “Small: 1”) of the P200 component of the aforementioned approximate expression, were used as threshold values. If the wavelet coefficient (x) is equal to or greater than the threshold value 5.2, the test subject is “Large”; if it is equal to or greater than the threshold value −1.0 but less than the threshold value 5.2, the test subject is “Middle”; and if it is less than the threshold value −1.0, the test subject is “Small”. Although middle values are illustrated as threshold values in the above example, this is only exemplary. Any value that is between “Large: 3” and “Middle: 2” and any value that is between “Middle: 2” and “Small: 1” may be used, other than middle values.

Based on the aforementioned approximate expression and threshold values, in the example of FIG. 6, it is possible to categorize a test subject into: type A; type B or C; or type D.

Now, the inventors' view concerning the above relationship will be discussed below. According to prior art document (Kiyoshi FUJISAWA et al., SIN SEIRISHINRIGAKU, (or “New Physiological Psychology”) vol. 1, p. 119, 1998), the N200 component (especially N2b) is supposed to reflect one's attentional focus responsive to an unexpected stimulation. Moreover, according to prior art document (Kiyoshi FUJISAWA et al., SIN SEIRISHINRIGAKU, vol. 2, p. 110, 1998), when the arousal level decreases, the a wave, which is a component from 8 Hz to 13 Hz of the electroencephalogram, also gradually decreases and eventually disappears, followed by an appearance of the θ wave having a low amplitude. Taking these into account, it may be considered that the test subjects whose N200 component were “Large” had a low arousal level during this experiment (that is, the near-α wave component decreased), and had a low concentration on executing the task of this experiment, and therefore experienced an attentional focus in response to a highlighting of the target option, as if responding to an unexpected stimulation, whereby the N200 component resulted.

On the other hand, it can be considered that the test subjects whose P200 component were “Large” had a high concentration on executing the task of this experiment, and therefore the near-α wave frequency component did not decrease in their wavelet coefficient, and large values were obtained especially in the time period from 200 milliseconds to 250 milliseconds.

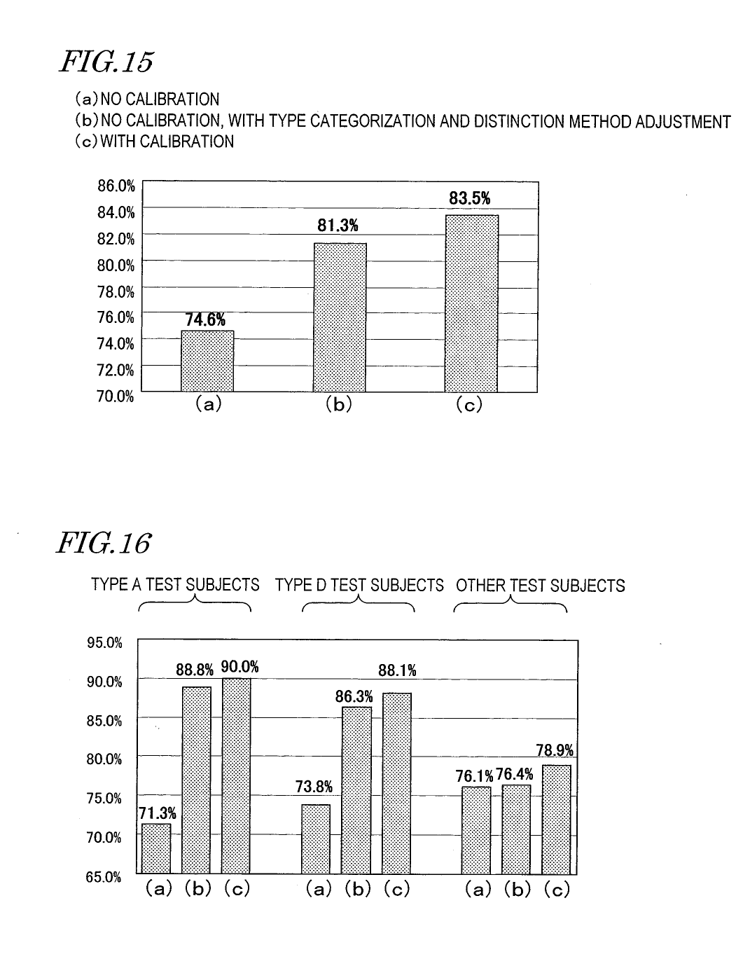

Note that, the actual levels of the N200 component and the P200 component may differ from the aforementioned result of type categorization. However, as will be described later based on results of distinction rate calculation in FIGS. 15 to 17, statistical speaking, the type categorization according to the present invention is very effective for maintaining and improving the distinction rate. Moreover, by simultaneously utilizing the power spectrum values of the frequency band shown in FIG. 8 and the wavelet coefficients of the time period and frequency band shown in FIG. 9, it becomes possible to perform a more detailed and accurate type categorization.

Next, with reference to the flowchart of FIG. 10, a procedure of processing by the category determination section for performing type categorization based on the aforementioned characteristic amounts will be described.

FIG. 10 shows a procedure of categorization process the category determination section 14.

At step S121, from the electroencephalogram IF section 13, the category determination section 14 receives electroencephalogram waveform data to be subjected to categorization. The electroencephalogram waveform data to be subjected to categorization is cut out by the electroencephalogram IF section 13 from an electroencephalogram signal which is measured by the electroencephalogram measurement section 12, and sent to the category determination section 14. In the example of portion (c) of FIG. 4, the category determination section 14 receives electroencephalogram waveform data 54a to 54d with respect to four highlighted options.

At step S122, with respect to all of the received electroencephalogram waveform data, the category determination section 14 extracts the following characteristic amounts, and calculates average values thereof: power spectrum values of the frequency band from about 8 Hz to about 15 Hz; and wavelet coefficients of the time period from 200 milliseconds to 250 milliseconds and the frequency band from about 8 Hz to about 15 Hz, which have been discussed in connection with the above experimental results.

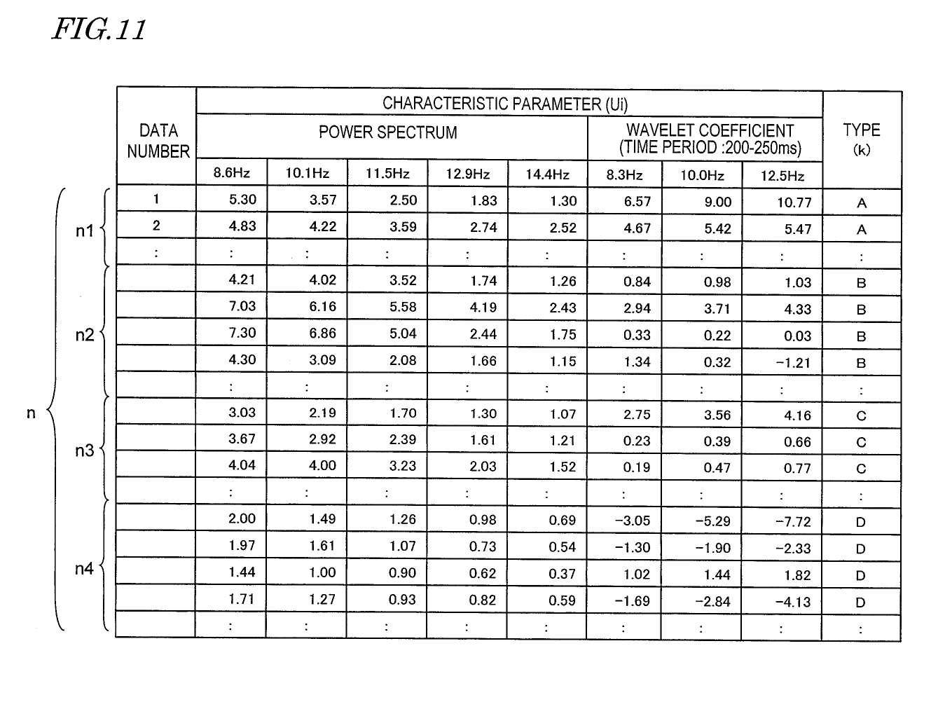

At step S123, the category determination section 14 reads reference data to be used for type categorization. FIG. 11 shows a portion of the reference data for type categorization which has been generated based on the aforementioned experimental results. The reference data for type categorization is composed of: a data number of each piece of electroencephalogram waveform data; characteristic parameters of the power spectrum and the wavelet coefficient; and a type which the particular piece of electroencephalogram waveform data belongs to. There are as many characteristic parameters of the power spectrum and as many characteristic parameters of the wavelet coefficient as there are samples in the zone from 8 Hz to 15 Hz. The number of samples is to be determined based on the sampling frequency, the time period to be cut out, etc., when measuring the electroencephalogram waveform data. It is assumed that the reference data shown in FIG. 11 is prestored in the category determination section 14. The values of the characteristic parameters to be actually described in FIG. 11 need to be prepared by performing the aforementioned experiment in advance.

At step S124, the category determination section 14 performs a type categorization by using the characteristic amounts extracted at step S122. The type categorization may be performed through a categorization based on the respective threshold values for the N200 component and P200 component as has been described with respect to the experimental results above, or through a discriminant analysis based on the type categorization data which has been read at step S123. Hereinafter, a discriminant analysis based on the type categorization data shown in FIG. 11 will be specifically described.

The category determination section 14 associates the four types of A to D of type categorization data with k=1, 2, 3, and 4, respectively, and by using characteristic parameters Ui (i=1 to 8), determines averages of characteristic parameters Ui for each of k types according to eq. 1 below.

Ūᵢ^{k}=(Ū₁^{k}, Ū₂^{k}, . . . , Ū₈^{k})′ [eq. 1]

The category determination section 14 determines a variance-covariance matrix S which is common to each type according to eq. 2 below.

$$S = ( {s}_{i , j} ) = \frac{1}{n - 4} {\sum }_{k = 1}^{4} {\sum }_{m = 1}^{nk} ( {U}_{i , m}^{k} - {\overline{U}}_{i}^{k} ) ( {U}_{j , m}^{k} - {\overline{U}}_{j}^{k} ) \qquad [ eq . 2 ]$$

In the above, n is a total number of data; nk is a number of data for each type; and i and j are integers of 1 to 8.

Assuming that average values of the power spectrum values of the frequency band from about 8 Hz to about 15 Hz extracted at step S122 and average values of the wavelet coefficients of the time period from 200 milliseconds to 250 milliseconds and the frequency band from about 8 Hz to about 15 Hz are Xi (i=1 to 8), a type k to which Xi belongs can be determined by determining a k which maximizes the linear function Zk below.

$${Z}_{k} = {X}^{′} \cdot {S}^{- 1} \cdot {\overline{U}}^{k} - \frac{1}{2} {\overline{U}}^{′ k} \cdot {S}^{- 1} \cdot {\overline{U}}^{k} \qquad [ eq . 3 ]$$

At step S125, the category determination section 14 sends the result of categorization at step S124 to the distinction method adjustment section 15.

A procedure of processing by the distinction method adjustment section 15 will be described with reference to the flowchart of FIG. 12.

At step S141, the distinction method adjustment section 15 receives a result of categorization by the category determination section 14.

At step S142, the distinction method adjustment section 15 reads distinction method adjustment data. It is assumed that the distinction method adjustment data is prestored in the distinction method adjustment section 15. The details thereof will be described below.

At step S143, in accordance with the result of categorization received at step S141, the distinction method adjustment section 15 selects a piece of data to be sent to the electroencephalogram IF section 13 as the result of adjustment, from among the distinction method adjustment data.

The aforementioned distinction method adjustment data to be read by the distinction method adjustment section 15 differs depending on the type of distinction method for a target option in the electroencephalogram IF section 13.

First, in the case of distinguishing a target option based on a zone average potential of the electroencephalogram waveform data in a given zone, the distinction method adjustment section 15 reads distinction method adjustment data which is shown in FIG. 13. FIG. 13 illustrates a look-up table showing weighting factors for the P300 component, the P200 component, and the N200 component with respect to each type. For example, if the result of type categorization is type A, weighting factors (1, 1, 0) for the P300 component, the P200 component, the N200 component with respect to type A are selected.

Next, in the case of distinguishing a target option based on a correlation coefficient value with respect to a template, the distinction method adjustment data to be read is electroencephalogram waveform data with respect to the target option, as shown by solid lines in FIGS. 7(a) to (d). For example, if the result of type categorization is type A, the electroencephalogram waveform data shown by a solid line in Portion (a) of FIG. 7 is selected as a template.

Lastly, in the case of distinguishing a target option based on a posterior probability value through a linear discriminant analysis or nonlinear discriminant analysis, the distinction method adjustment data to be read is model data which is prepared for each type. FIG. 14 shows an example model data of type A, where portion (a) shows electroencephalogram waveform data (number of data: 80) for a target option, and portion (b) shows electroencephalogram waveform data (number of data: 240) for non-target options. If the result of type categorization is type A, the data of FIG. 14 are to be selected as model data.

At step S144, the distinction method adjustment section 15 sends the data selected at step S143i to the electroencephalogram IF section 13 as the result of adjustment.

Now, the process of distinguishing a target option by the electroencephalogram IF section 13 will be described again (step S67 in FIG. 3). In response to the result of adjustment by the distinction method adjustment section 15, the following process is performed.

First, in the case of distinguishing a target option based on a zone average potential of electroencephalogram waveform data in a given zone, a calculation expressed by eq. 4 below is performed for each piece of electroencephalogram waveform data for a highlighted option.

E=W_{P3}·P_{P3}+W_{P2}·P_{P2}·W_{N2}·P_{N2}

Herein, Wp3, Wp2, and Wn2 are weighting factors for the P300 component, the P200 component, and the N200 component, respectively, received from the distinction method adjustment section 15. FIG. 13 shows these weighting factors. For example, if the category determination section 14 categorizes the electroencephalogram waveform of a user to be type A, i.e., if it determines that the electroencephalogram waveform data for the target option will have a large P200 component and a small N200 component, the distinction method adjustment section 15 applies weighting in favor of the P200 component by setting the above weighting factors to (1, 1, 0).

Similarly, when the category determination section 14 categorizes the electroencephalogram waveform of a user to be type D, i.e., if it determines that the electroencephalogram waveform data for the target option will have a small P200 component and a large N200 component, the distinction method adjustment section 15 applies weighting in favor of the N200 component by setting the above weighting factors to (1, 0, 1). Pp3, Pp2, and Pn2 are the P300 component (average potential from 300 milliseconds to 500 milliseconds), P200 component (average potential from 200 milliseconds to 300 milliseconds), N200 component (average potential from 100 milliseconds to 200 milliseconds), respectively, and E represents an evaluation value. Since the N200 component is characterized in that it appears as a negative potential in response to a target option, it is subtracted in the above expression so as to become reflected in the evaluation value E. An evaluation value E is calculated from the electroencephalogram waveform data for each highlighted option, and an option whose value is the largest is distinguished as the target option.

Next, in the case of distinguishing a target option based on a correlation coefficient value with respect to a template, a correlation coefficient of the electroencephalogram waveform data for each highlight option with respect to a template which is received from the distinction method adjustment section 15, e.g., a Pearson product-moment correlation coefficient, is determined, and an option whose value is the largest is distinguished as the target option.

Lastly, in the case of distinguishing a target option based on a posterior probability value through a linear discriminant analysis or nonlinear discriminant analysis, the electroencephalogram waveform data for each highlight option is subjected to a linear discriminant analysis or a nonlinear discriminant analysis based on model data which is received from the distinction method adjustment section 15. Specifically, a posterior probability which represents a likelihood of being a target option by using Bayesian estimation is determined, and an option whose value is the largest is distinguished as the target option.

With the above-described method, based on the result of adjustment to the distinction method which is made by the distinction method adjustment section 15, it becomes possible to distinguish a target option from among a plurality of options.

The processing by the category determination section 14 and the distinction method adjustment section 15 described above may be automatically performed every time a user uses an electroencephalogram interface, or may be performed upon a user's instruction, and the result of adjustment therefrom may be retained in the electroencephalogram IF section 13.

The effects obtained by the above-described embodiment of the present invention will be specifically described based on results of trial calculations of a distinction rate for a target option.

Trial calculations of a distinction rate were performed based on the aforementioned experimental results (results of an experiment involving 13 test subjects where one among four options was selected by using electroencephalograms). A linear discriminant analysis was used for the type categorization by the category determination section 14 shown in FIG. 2, where both of the power spectrum and the wavelet coefficient of the electroencephalogram waveform data were used as characteristic amounts. A linear discriminant analysis was also used for the distinction of a target option by the electroencephalogram IF section 13 shown in FIG. 2, where an average potential of electroencephalogram waveform data taken every 25 milliseconds was used as a characteristic amount.

An objective of these trial calculations of a distinction rate was to make a comparison of distinction rates under the following three conditions, thus to confirm the effects of the present invention: (a) not performing a calibration for each test subject; (b) not performing a calibration, but performing a type categorization and an adjustment of the distinction method according to the present invention; and (c) performing a calibration for each test subject. Therefore, as the model data to be used for the distinction of a target option, experimental results from all test subjects were used in case (a), thus employing model data which was common to all test subjects. In case (b), a type categorization according to the present invention was performed, and model data according to the result of categorization was used; for example, if a test subject was categorized as type A, the experimental results from the test subjects belonging to type A (test subjects 01 and 08 in the example of FIG. 5) were used as the model data. In case (c), model data for each test subject was used; for example, the experimental result from test subject 01 was used as the model data in the case of test subject 01. However, in all of the aforementioned conditions, the data to be evaluated was always excluded from the model data in performing a distinction of a target option, thus making an evaluation by the so-called leave-1-out method.

FIG. 15 shows an average value, across all test subjects, of a distinction rate for a target option under each of the three conditions. The distinction rate is lowest in case (a) of performing no calibration (74.6%), and highest in case (c) of performing a cumbersome and complicated calibration (83.5%). It can be seen that in case (b) of employing the present invention, an accuracy which is close to that of case (c) (with a calibration) is obtained, although a calibration for each test subject is not performed (81.3%).

FIG. 16 shows, as a breakdown of FIG. 15, the respective distinction rates of the test subjects of type A, the test subjects of type D, and the other test subjects. It can be seen from FIG. 16 that the effects of the present invention are evident with the test subjects of type A and the test subjects of type D. In other words, in case (b) of employing the present invention, the distinction rate is greatly improved as compared to case (a), and about the same accuracy of distinction is maintained as in case (c), although a complicated calibration for each test subject is not performed.

Thus, as is clear from FIG. 15(b) and FIG. 16(b), by introducing the electroencephalogram distinction method adjustment apparatus 2 of the present invention in the electroencephalogram interface system 1, the trouble of an advance calibration, which has conventionally been a burden on the user, can be eliminated while maintaining a high accuracy of distinction.

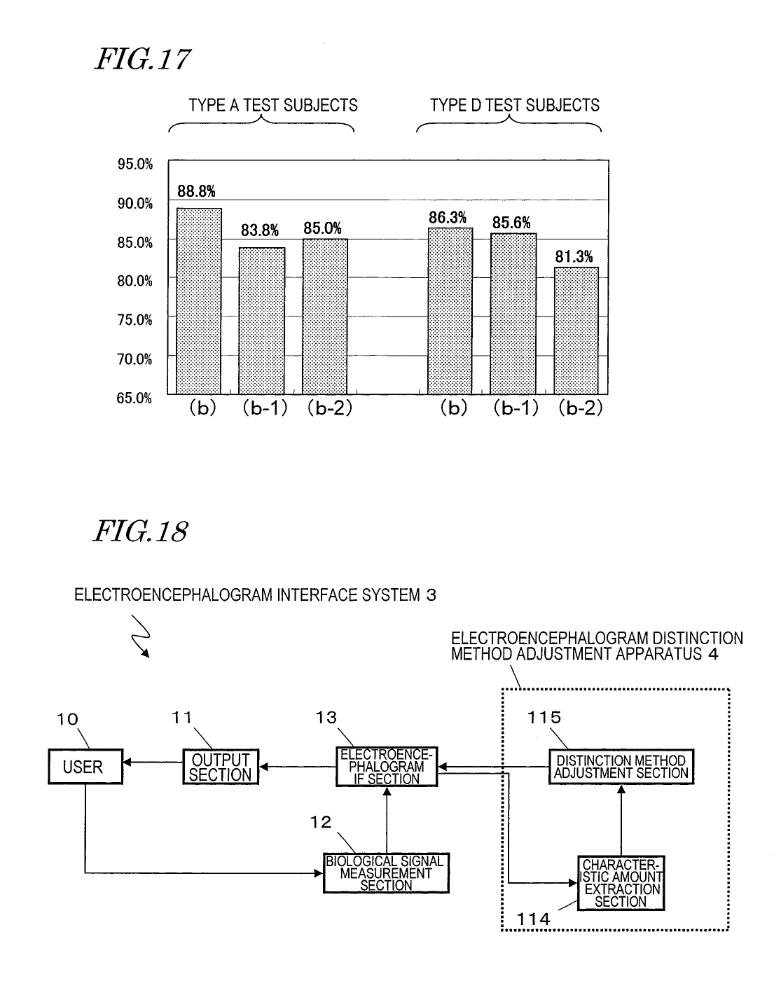

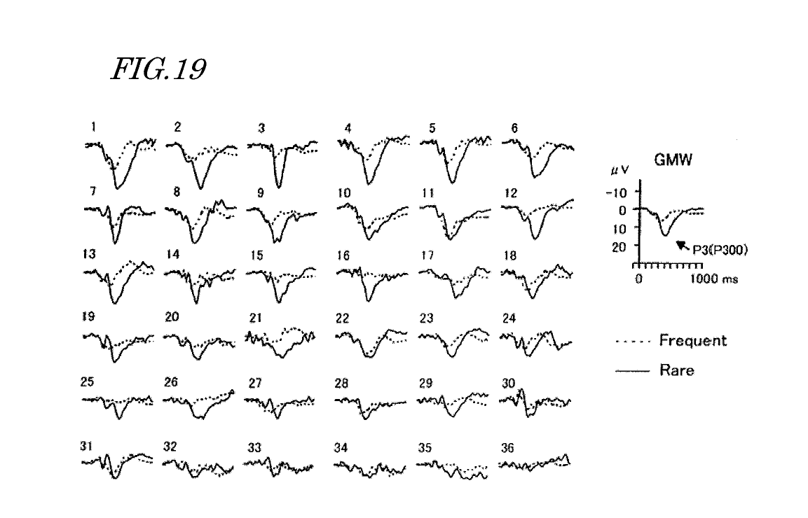

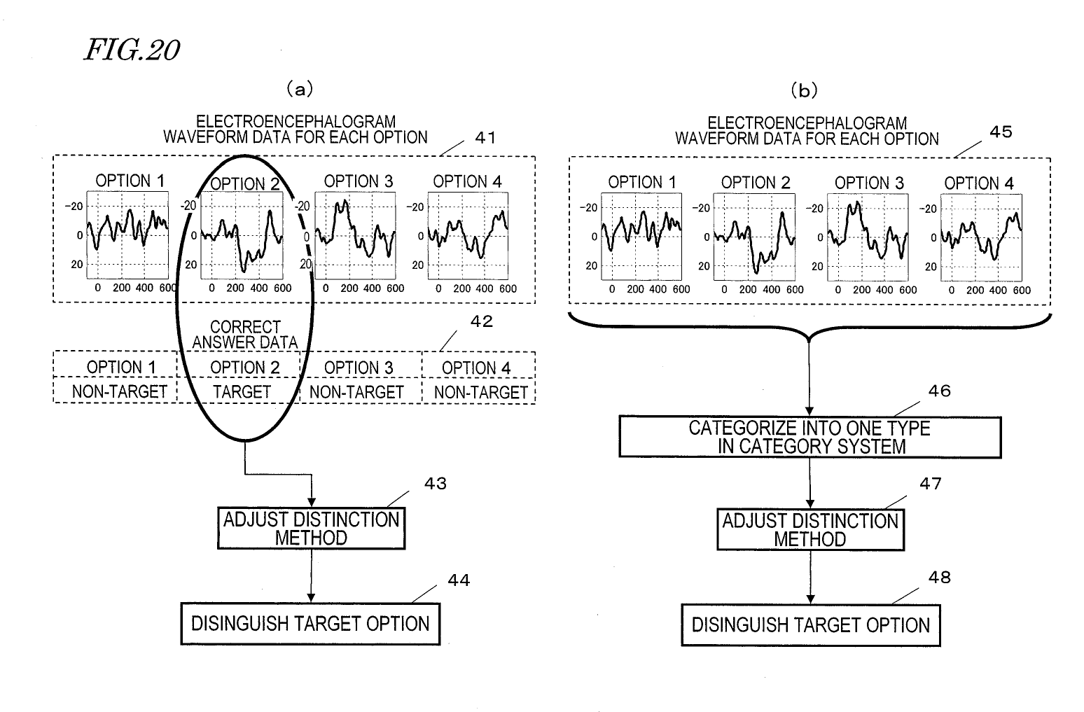

Furthermore, FIG. 17 the distinction rates of the test subjects of type A and type D with respect to the three conditions where the characteristic amounts to be used for type categorization are: (b) both power spectrum values and wavelet coefficients; (b-1) only power spectrum values; and (b-2) only wavelet coefficients. Note that FIG. 17(b) and FIG. 16(b) pertain to the same evaluation. From FIG. 17, it can be seen that, in case (b-1) of employing only power spectrum values and case (b-2) of employing only wavelet coefficients, the distinction rate is somewhat lower than in case (b) of employing both, but is greatly improved over the case of FIG. 16(a), without even performing a calibration. Thus, it can be seen that effects can be obtained with only one of the power spectrum and the wavelet coefficient of electroencephalogram waveform data.

The present embodiment is very effective in the case where categorization is made based on an event-related potential for each option which is obtained with a small number of stimulations (e.g., about 1 to 3) and on the aforementioned N200 component and P200 component. FIG. 15 to FIG. 17 show that this is particularly evident in the case where categorization is made based on average values of power spectrum values of a frequency band and/or average values of wavelet coefficients of a frequency band.

Therefore, the characteristic amounts to be used for the type categorization may be both of the power spectrum and the wavelet coefficient of electroencephalogram waveform data as described above, or either one of them. In the case of only utilizing power spectrum values, a categorization is made as to whether the N200 component is “Large” or “Small”; in the example of FIG. 6, a categorization will be made into the two types of types C and D or types A and B. Similarly, in the case of only utilizing wavelet coefficients, a categorization is made as to whether the P200 component is “Large”, “Middle”, or “Small”; in the example of FIG. 6, a categorization will be made into the three types of type A, types B and C, or type D.

With the construction and procedure of processing according to the present embodiment, in a system having an interface for distinguishing from among a plurality of options an option which a user wishes to select by utilizing an electroencephalogram, by using characteristic amounts which are common to the electroencephalogram waveform data corresponding to all options, or more specifically, by using average values of the power spectrum values of the frequency band from about 8 Hz to about 15 Hz and average values of the wavelet coefficients of the time period from 200 milliseconds to 250 milliseconds and the frequency band from about 8 Hz to about 15 Hz, a categorization into one type in a previously-prepared category system is made. By performing a process of adjustment for arriving at an optimum distinction method according to the result of categorization, it is possible to eliminate the user's burden of complicated calibration, and maintain a high accuracy of distinction for electroencephalograms.

With respect to the above-described embodiment, any process that was described by employing a flowchart can be implemented as a program to be executed by a computer. Such a computer program may be distributed on the market in the form of a product recorded on a storage medium, such as a CD-ROM, or transmitted via telecommunication lines such as the Internet. All or some of the constituent elements composing the distinction method adjustment apparatus and the electroencephalogram IF section may be implemented as a general-purpose processor (semiconductor circuit) executing a computer program. Alternatively, they may be implemented as a special processor in which such a computer program and a processor are integrated. A computer program which realizes the functions of the electroencephalogram distinction method adjustment apparatus may be executed by a processor which executes a computer program for realizing the functions of the electroencephalogram IF section, or by any other processor within the electroencephalogram interface system.

Moreover, although the electroencephalogram distinction method adjustment apparatus 2 is provided in the output section (television set) 11 together with the electroencephalogram IF section 13 in the present embodiment, this is only exemplary. One or both of them may be provided outside the television set.

### Embodiment 2

In Embodiment 1, by using a characteristic amount(s) which is common to the electroencephalogram waveform data corresponding to all options, a characteristic feature of the electroencephalogram of an individual person is categorized into one type in the classified category system shown in FIG. 6. Then, a process of making an adjustment for arriving at an optimum distinction method is performed based on the result of categorization (step 66 in FIG. 3).

As described in Embodiment 1, it has been found that a characteristic amount can be extracted from the electroencephalogram waveform with respect to any option. In view of this, if a characteristic amount can be extracted from the electroencephalogram waveform with respect to any option, it should be clear that a characteristic amount can be extracted more easily and the accuracy will be improved over the conventional level by using the electroencephalogram waveforms with respect to two or more options among all options.

Therefore, in the present embodiment, electroencephalogram waveforms with respect to all options are not used. Instead, electroencephalogram waveforms with respect to some of the options (at least two among all options, where “all” options consist of three or more) are used. Moreover, without employing a type categorization as shown in FIG. 6, it is determined as to whether an N200 characteristic amount or a characteristic amount P200 characteristic amount exists in the electroencephalogram waveforms with respect to the non-all options, and weighting is applied to that characteristic amount in determining a target option.

FIG. 18 shows a functional block construction of an electroencephalogram interface system 3 according to the present embodiment. The electroencephalogram interface system 3 includes an output section 11, an electroencephalogram measurement section 12, an electroencephalogram IF section 13, and an electroencephalogram distinction method adjustment apparatus 4. The differences from the electroencephalogram interface system 1 of Embodiment 1 are the construction and operation of the electroencephalogram distinction method adjustment apparatus.

The electroencephalogram distinction method adjustment apparatus 4 of the present embodiment is composed of a characteristic amount extraction section 114 and a distinction method adjustment section 115. Hereinafter, only the differences from Embodiment 1 will be described. The constituent elements of Embodiment 2 are identical to the constituent elements of Embodiment 1 except for those mentioned otherwise, and their descriptions will be omitted.

From the electroencephalogram signals after respective options are presented, the characteristic amount extraction section 114 selects electroencephalogram signals with respect to two or more options. The characteristic amount extraction section 114 prestores reference data, and extracts a characteristic amount which is common to the reference data and the selected electroencephalogram signals.

The distinction method adjustment section 115 applies weighting to the characteristic amount extracted by the characteristic amount extraction section 114, and adjusts the distinction method for the electroencephalogram signal with respect to an option selected by the user 10. Then, it sends the result of adjustment to the electroencephalogram IF section 13. As a result, the distinction method for distinguishing a component of the event-related potential in the electroencephalogram IF section 13 is changed.

The flowchart of FIG. 3 is generally applicable to the processing by the electroencephalogram interface system 3 of the present embodiment. However, step S66 differs in the following respect.

In the present embodiment, at step S66, the characteristic amount extraction section 114 of the electroencephalogram distinction method adjustment apparatus 4 selects electroencephalogram signals corresponding to two or more options from among electroencephalogram signals which have been obtained with respect to three or more options. Furthermore, the characteristic amount extraction section 114 performs an extraction from the selected electroencephalogram waveforms, and determines which one of an N200 characteristic amount or a P200 characteristic amount is contained. The characteristic amount can be determined from the power spectrum values of the frequency band from about 8 Hz to about 15 Hz, or wavelet coefficients of the time period from 200 milliseconds to 250 milliseconds and the frequency band from about 8 Hz to about 15 Hz.

Note that, as shown in FIG. 6, it will never be the case that the P200 component and the N200 component are both Large or both Small. Therefore, the characteristic amount extraction section 114 can surely determine whether the selected electroencephalogram waveform contains an N200 characteristic amount or a P200 characteristic amount. In the present embodiment, the characteristic amount extraction section 114 retains the reference data shown in FIG. 11, and determines which one of the N200 or P200 characteristic amount is contained.

The distinction method adjustment section 115 makes an adjustment of the distinction method in the electroencephalogram IF section 13 so as to apply weighting according to the determined characteristic amount. As a result, when distinguishing an electroencephalogram signal with respect to an option selected by the user at step S67 of FIG. 3, it is possible to distinguish a target option. Weighting means applying weighting factors as shown in FIG. 13 to an electroencephalogram signal at the time of electroencephalogram distinction.

As described above, in the present embodiment, the electroencephalogram signal is not categorized into types A to D as shown in FIG. 6. Therefore, processes concerning categorization such as steps S123 and S124 in FIG. 10 do not need to be performed.

Note that, the processing of the present embodiment can also be implemented as a program to be executed by a computer. The descriptions of such a program will be identical to the descriptions of the program in Embodiment 1, and therefore are omitted.

An electroencephalogram distinction method adjustment apparatus according to the present invention and an electroencephalogram interface system incorporating the apparatus are useful for improving the manipulability of any device whose distinction method needs to be improved by allowing individual differences in electroencephalograms to be reflected thereupon, e.g. an information device or audio-video device in which a device manipulation interface utilizing electroencephalograms is incorporated, and any system that is used by an indefinite number of users, e.g., a ticket vending machine at a train station or a bank ATM.

While the present invention has been described with respect to preferred embodiments thereof, it will be apparent to those skilled in the art that the disclosed invention may be modified in numerous ways and may assume many embodiments other than those specifically described above. Accordingly, it is intended by the appended claims to cover all modifications of the invention that fall within the true spirit and scope of the invention.

What is claimed is:

1. In an electroencephalogram interface system having
an output section for presenting on a screen a plurality of options related to device operations and highlighting each option,
an electroencephalogram measurement section for measuring an electroencephalogram signal from a user, and an electroencephalogram interface section for, by using a previously determined distinction method, distinguishing an event-related potential with respect to an option which the user wishes to select from an event-related potential of the electroencephalogram signal based on a timing of highlighting each option as a starting point, and determining an operation of a device,
an adjustment apparatus for an electroencephalogram distinction method to be used for adjusting the distinction method in the electroencephalogram interface section, wherein,
the distinction method is a method of distinguishing a component of the event-related potential based on whether the electroencephalogram signal satisfies a predetermined criterion or not, the apparatus comprising:
a category determination section which prestores reference data for classifying a characteristic feature of an electroencephalogram signal, for determining which one of a plurality of classified categories the measured electroencephalogram signal belongs to by using the reference data and a characteristic amount which is common to electroencephalogram signals with respect to two or more options presented by the output section; and
a distinction method adjustment section for, based on a result of categorization, adjusting the distinction method for an electroencephalogram signal with respect to the option selected by the user.

2. The adjustment apparatus of claim 1, wherein the electroencephalogram signals with respect to the two or more options presented by the output section used by the category determination section are electroencephalogram signals with respect to all of the plurality of options that are presented by the output section.

3. The adjustment apparatus of claim 1, wherein, as the characteristic amount which is common to electroencephalogram signals with respect to all of the plurality of options, the category determination section retains an average value of power spectrum values of a predetermined frequency band and/or an average value of wavelet coefficients of a predetermined time period and frequency band of the electroencephalogram signals with respect to the two or more options presented by the output section.

4. The adjustment apparatus of claim 3, wherein the category determination section determines an amplitude of an N200 component of the electroencephalogram signal by using an average value of power spectrum values in a frequency band from 8 Hz to 15 Hz.

5. The adjustment apparatus of claim 3, wherein the category determination section determines an amplitude of a P200 component of the electroencephalogram signal by using an average value of wavelet coefficients of a time period from 200 milliseconds to 250 milliseconds and a frequency band from 8 Hz to 15 Hz.

6. The adjustment apparatus of claim 1, wherein, based on the result of categorization, the distinction method adjustment section adjusts weighting factors for a P300 component, a P200 component, and an N200 component of an electroencephalogram signal, the weighting factors being used for distinguishing an electroencephalogram signal with respect to the option selected by the user.

7. The adjustment apparatus of claim 1, wherein, for each of the plurality of classified categories, the distinction method adjustment section retains a template to be used for distinguishing an electroencephalogram signal with respect to the option selected by the user, and the distinction method adjustment section adjusts the distinction method for the electroencephalogram signal by using a template corresponding to the result of categorization.

8. The adjustment apparatus of claim 1, wherein, by adopting model data to be used for distinguishing an electroencephalogram signal with respect to the option selected by the user according to the result of categorization, the distinction method adjustment section adjusts the distinction method for the electroencephalogram signal.

9. In an electroencephalogram interface system having
an output section for presenting on a screen a plurality of options related to device operations and highlighting each option,
an electroencephalogram measurement section for measuring an electroencephalogram signal from a user, and
an electroencephalogram interface section for, by using a previously determined distinction method, distinguishing an event-related potential with respect to an option which the user wishes to select from an event-related potential of the electroencephalogram signal based on a timing of highlighting each option as a starting point, and determining an operation of a device,
an adjustment method to be used for adjusting the distinction method in the electroencephalogram interface section, wherein,
the distinction method is a method of distinguishing a component of the event-related potential based on whether the electroencephalogram signal satisfies a predetermined criterion or not, the method comprising the steps of:
preparing reference data for classifying a characteristic feature of an electroencephalogram signal;
determining which one of a plurality of classified categories the measured electroencephalogram signal belongs to by using the reference data and a characteristic amount which is common to electroencephalogram signals with respect to two or more options presented by the output section; and
based on a result of categorization, adjusting the distinction method for an electroencephalogram signal with respect to the option selected by the user.

10. In an electroencephalogram interface system having
an output section for presenting on a screen a plurality of options related to device operations and highlighting each option,
an electroencephalogram measurement section for measuring an electroencephalogram signal from a user, and
an electroencephalogram interface section for, by using a previously determined distinction method, distinguishing an event-related potential with respect to an option which the user wishes to select from an event-related potential of the electroencephalogram signal based on a timing of highlighting each option as a starting point, and determining an operation of a device,
a computer program embodied in a computer-readable medium and to be used for adjusting the distinction method in the electroencephalogram interface section, wherein,
the distinction method is a method of distinguishing a component of the event-related potential based on whether the electroencephalogram signal satisfies a predetermined criterion or not, the computer program causing a computer which is provided in the electroencephalogram interface system to execute the steps of:
prestoring reference data for classifying a characteristic feature of an electroencephalogram signal;
determining which one of a plurality of classified categories the measured electroencephalogram signal belongs to by using the reference data and a characteristic amount which is common to electroencephalogram signals with respect to two or more options presented by the output section; and
based on a result of categorization, adjusting the distinction method for an electroencephalogram signal with respect to the option selected by the user.

11. In an electroencephalogram interface system having
an output section for presenting on a screen a plurality of options related to device operations and highlighting each option,
an electroencephalogram measurement section for measuring an electroencephalogram signal from a user, and
an electroencephalogram interface section for, by using a previously determined distinction method, distinguishing an event-related potential with respect to an option which the user wishes to select from an event-related potential of the electroencephalogram signal based on a timing of highlighting each option as a starting point, and determining an operation of a device,
an adjustment apparatus for an electroencephalogram distinction method to be used for adjusting the distinction method in the electroencephalogram interface section, wherein,
the distinction method is a method of distinguishing a component of the event-related potential based on whether the electroencephalogram signal satisfies a predetermined criterion or not, the apparatus comprising:
a characteristic amount extraction section for
(i) selecting electroencephalogram signals with respect to two or more options from among the electroencephalogram signals with respect to the options, and
(ii) prestoring reference data, and extracting a characteristic amount which is common to the reference data and the selected electroencephalogram signals; and
a distinction method adjustment section for adjusting the distinction method for an electroencephalogram signal with respect to the option selected by the user so as to apply weighting according to the extracted characteristic amount in distinguishing the electroencephalogram signal with respect to the option selected by the user.

12. In an electroencephalogram interface system having
an output section for presenting on a screen a plurality of options related to device operations and highlighting each option,
an electroencephalogram measurement section for measuring an electroencephalogram signal from a user, and
an electroencephalogram interface section for, by using a previously determined distinction method, distinguishing an event-related potential with respect to an option which the user wishes to select from an event-related potential of the electroencephalogram signal based on a timing of highlighting each option as a starting point, and determining an operation of a device,
an adjustment method to be used for adjusting the distinction method in the electroencephalogram interface section, wherein,
the distinction method is a method of distinguishing a component of the event-related potential based on whether the electroencephalogram signal satisfies a predetermined criterion or not, the method comprising the steps of:
selecting electroencephalogram signals with respect to two or more options from among the electroencephalogram signals with respect to the options;
prestoring reference data, and extracting a characteristic amount which is common to the reference data and the selected electroencephalogram signals; and
adjusting the distinction method for an electroencephalogram signal with respect to the option selected by the user so as to apply weighting according to the extracted characteristic amount in distinguishing the electroencephalogram signal with respect to the option selected by the user.

13. In an electroencephalogram interface system having
an output section for presenting on a screen a plurality of options related to device operations and highlighting each option,
an electroencephalogram measurement section for measuring an electroencephalogram signal from a user, and
an electroencephalogram interface section for, by using a previously determined distinction method, distinguishing an event-related potential with respect to an option which the user wishes to select from an event-related potential of the electroencephalogram signal based on a timing of highlighting each option as a starting point, and determining an operation of a device,
a computer program embodied in a computer-readable medium and to be used for adjusting the distinction method in the electroencephalogram interface section, wherein,
the distinction method is a method of distinguishing a component of the event-related potential based on whether the electroencephalogram signal satisfies a predetermined criterion or not, the computer program causing a computer which is provided in the electroencephalogram interface system to execute the steps of:
selecting electroencephalogram signals with respect to two or more options from among the electroencephalogram signals with respect to the options;
prestoring reference data, and extracting a characteristic amount which is common to the reference data and the selected electroencephalogram signals; and
adjusting the distinction method for an electroencephalogram signal with respect to the option selected by the user so as to apply weighting according to the extracted characteristic amount in distinguishing the electroencephalogram signal with respect to the option selected by the user.
